(19) United States
(12) Patent Application Publication
(10) Pub. No.: US 2023/0287408 A1
(43) Pub. Date: Sep. 14, 2023

(54) NUCLEIC ACID CONSTRUCTS FOR DELIVERING POLYNUCLEOTIDES INTO EXOSOMES

(71) Applicant: ReNeuron Limited, Pencoed, Bridgend, Wales (GB)

(72) Inventors: Randolph CORTELING, Pencoed (GB); Ruben DEOGRACIAS, Pencoed (GB)

(21) Appl. No.: 17/785,801

(22) PCT Filed: Dec. 18, 2020
(86) PCT No.: PCT/GB2020/053284

§ 371 Date: Jun. 15, 2022

(30) Foreign Application Priority Data
Dec. 20, 2019 (GB) 1919021.4

(51) Int. Cl. C12N 15/113; C12N 15/88; C12N 15/86

(52) U.S. Cl. CPC C12N 15/113; C12N 15/86; C12N 15/88; C12N 2310/141; C12N 2310/531; C12N 2310/533; C12N 2320/32; C12N 2740/15043

(57) ABSTRACT

The invention delivers exogenous nucleotide sequences into exosomes using structural and regulatory characteristics identified in the miRNA molecules MIR21, pri-miR-21 and pre-miR- 21. In particular, the invention relates to pre-miRNA for targeting an exogenous nucleotide sequence to an exosome, wherein the pre-miRNA comprises an exogenous nucleotide sequence and a stem-loop structure, wherein the stem comprises at least one wobble pair. The invention also provides nucleic acid cassettes, vectors and cells comprising the engineered pre-miRNA, methods of loading exosomes and the resulting loaded exosomes. The loaded exosomes can be used to deliver an exogenous nucleotide sequence to a target cell, for example in therapy.

### REFERENCE TO A SEQUENCE LISTING

The Sequence Listing written in file 2021-01-05-P78470WO-SeqListing-ST25.txt created on Dec. 12, 2022, 10,229 bytes, machine format IBM-PC, MS-Windows operating system, in accordance with 37 C.F.R. §§ 1.821 to 1.825, is hereby incorporated by reference in its entirety for all purposes.

### FIELD OF THE INVENTION

This invention relates to nucleic acid constructs that are able to deliver nucleotide sequences into exosomes, and methods of using these constructs to produce exosomes comprising a nucleotide sequence of interest.

### BACKGROUND OF THE INVENTION

Exosomes are membrane-bound vesicles that originate in the endosomal compartment and are secreted from cells when Multivesicular Bodies (MVBs) fuse with the plasma membrane. Exosomes typically have a diameter between 30 and 120 nm. Exosomes can contain many types of biomolecule, including proteins, carbohydrates, lipids and nucleic acids. Exosome biogenesis involves the enrichment of their membrane with cholesterol, ceramide and other lipids typically found in detergent-resistant membranes of cells. Proteins are also embedded in the exosome membrane, such as tetraspanins and other transmembrane proteins and receptors that may play a role in their sorting and biological activity (Colombo et al. 2014).

The lumen of exosomes contains specific proteins and nucleic acids, such as microRNAs (miRNAs), which confer upon the exosomes the capacity to modulate transcription and translation in target cells. The repertoire of miRNAs within exosomes appears to reflect the physiological state of the producer cell, with selected miRNAs shuttled into the exosomes. It has also been observed that secretion of exosome-encapsulated or exosome-associated miRNAs plays a role in effecting the paracrine activities of the cells that produced them. This mechanism may allow cells to exert local and distant control, both in normal conditions and in response to a local stimulus such as an infection, nutrient starvation, or hypoxia.

Loading exogenous cargo molecules such as miRNAs of interest into exosomes has been suggested as a useful way to harness their ability to deliver signals between cells. One method is to introduce biological molecules of interest directly into harvested exosomes. However, despite this being possible at small scales using techniques such as electroporation and lipofection, these techniques have not proven to be repeatable, scalable or reliable at a large scale. Moreover, these techniques may even compromise the structure of the exosome (Janas, et al. 2015).

miRNAs have been shown to be selectively shuttled by cells into exosomes. Around 60% of miRNA coding sequences are located in intra- or intergenic regions and therefore expression depends on the regulatory sequences and transcription factors that regulate the expression of the primary genes. However, a smaller proportion of miRNA coding regions are located in intergenic regions and contain their own promoter sequences, enhancers and repressors. Therefore, the expression of these miRNAs depend on an additional level of regulation to control specifically their correct expression in a cell-dependent and stimulus-dependent manner that is reliant on promoter mediated activation governed by other genes. In both cases, miRNAs are initially transcribed embedded within long precursors called pri-miRNAs (“primary miRNAs”), wherein the functional miRNA is surrounded by sequences necessary for its efficient and correct processing and cleavage by a protein complex containing the enzyme Drosha. The correct processing in the nucleus by Drosha forms a shorter precursor known as pre-miRNA. This pre-miRNA typically forms a stem-loop structure, which is then transported from the nucleus to the cytoplasm where it is recognised by the protein complex RISC, which includes the RNase DICER and Argonaute (Ago) proteins. Dicer cleaves the loop part of the pre-miRNA structure to form a mature double-stranded miRNA, and one of the strands (known as the passenger strand) is degraded or discarded, likely by Ago, leaving a single-stranded guide strand within RISC. The RISC complex is then able to silence gene expression of messenger RNAs comprising a sequence complementary to the single stranded miRNA guide strand. Both the length of the hairpin and the size of the loop in a pre-miRNA has been suggested to be critical for the correct cleavage by DICER and for the localisation of the guide miRNA strand (Tsutsumi, et al. 2011).

Currently, it is unclear how miRNAs that are shuttled into exosomes escape cytoplasmic processing by RISC in the cell that produces the exosomes. Ago proteins generally are not located in exosomes, which suggests a specific mechanism for the sorting miRNAs into exosomes. Up-shuttling of miRNAs into exosomes has been shown to require the interaction of pre-miRNAs or mature miRNAs with a different set of RNA binding proteins (RBPs), which have high affinity for the ceramides that are located in the endosomal membrane during MVB formation (Villarroya-Beltrei, et al. 2013). However, different cell and microRNA combinations have reported different RBPs as chaperones. There appears to be no consensus on which of the RBPs are responsible for the selective shuttling of miRNAs into exosomes and it appears that different RBPs are employed from cell to cell.

The identification of RBP-interacting motifs involved in sorting miRNAs into exosomes has opened the possibility of genetically-modifying cells to produce exosomes that carry an exogenous nucleotide of interest. However, although some cell/microRNA-specific shuttling RNA binding proteins and sequence tags have been identified, no universally applicable and accepted mode of miRNA packaging into exosomes has yet been identified. Some groups have suggested that small sequences termed EXOmotifs (GCCG, UGAC, UCCG, GGAC, GGCG and UGCC) could be responsible for sorting miRNAs into the exosome. Despite its presence in many natural occurring miRNAs, the efficiency of loading the modified miRNAs with EXOmotifs into exosomes is variable, dependent on the cell and target sequence to be loaded, and in some cases shows no benefit (Villarroya-Beltrei, et al. 2013). Others have used the 25 nucleotide sequence found in the 3′-untranslated region (3′UTR) of many mRNAs targeted by miRNA, which was observed to help up-shuttle messenger RNAs (mRNAs) and therefore possibly miRNA within microvesicles secreted from glioblastoma multiforme cell lines. These 25 nucleotide sequences are termed ‘zipcodes’ and all utilise a CUGCC core sequence present on a stem-loop structure (Bolukbasi, et al. 2012).

WO-A-2018/209182 describes particular RNA sequences referred to as “EXO-codes” that selectively sort to exosomes and can deliver a cargo to the exosomes. Sutaria et al (2017) describe the use of the TAT peptide/HIV-1 transactivation response (TAR) RNA interacting peptide to enhance loading of a TAR RNA loop into extracellular vesicles. WO-A-2019/226603, WO-A-2019/204733 and WO-A-2015/183667 describe hybrid tRNA-miRNA stem-loop structures. These engineered chimeric nucleic acids can be packaged into synthetic liposomes or nanoparticles for delivery to cells or patients.

In summary, there is no clear unifying sequence or determinant for loading exogenous nucleotide sequences into exosomes during exosome biogenesis, that work efficiently enough for general utility across cell types (Yoo, et al. 2018).

In WO-A-2017/054085, a method of loading exosomes with miRNA using expression vectors containing a modified pre-miR-451 structural mimic is described. US 8,273,871 and Yang, et al. 2010 also describe unusual properties of pre-miR-451. This technology requires a cell line with low expression levels of Ago, or preferably no Ago, and one that preferably naturally produces high levels of miR-451 and/or a miRNA that does not require cleavage by DICER. In vectors described by WO-A-2017/054085, the stem miRNA and the loop are replaced by designed nucleotide sequences that maintain the length of the pre-miRNA independently of the sequence. The authors maintain that keeping the correct length of the miRNA, independently of the sequence of the loop, should maintain the cleavage sites for processing the pre-miRNA. Contrary to this, Gu et al. (Cell 151. 900-911 Nov. 9, 2012), reported that the sequence of the loop is not important for the correct processing of the miRNA, but the distance between the loop and the sequence of the miRNA is critical to produce not only the correct cleavage of the pre-miRNA, to release the miRNA, but also to avoid any off-target effect due to a non-specific recognition of the incorrect form of the miRNA generated. Therefore, the correct processing of the miRNA is a fundamental step to obtain a molecule not only with the correct length but also the correct function.

Moreover, accurate recognition of the RNA helices (A-form or B-form) by RBPs to form stable RNA-protein complexes may depend on the non-Watson-Crick wobble G-U pairs that are observed in naturally occurring pre-miRNA. Studies on the three-dimensional structure of large RNA-protein complexes have probed that Wobble G-U pairs are key structural elements, distorting the RNA deep groove to allow the native folding of the RNA and its recognition by RBPs. These three-dimensional studies have also identified other base-pairs that give a different spatial conformation to miRNAs to be recognised by specific RBPs (Arachchilage et al. 2015). Despite this, none of the previously described approaches have considered the functional importance of the non-canonical G-U wobble pairs in parts of the pre-miRNA such as in the loop structure or in the pre-miRNA sequence.

There remains a need to be able to load nucleotide sequences of interest into exosomes, in a predictable, efficient and controllable manner.

### SUMMARY OF THE INVENTION

The invention is based on the surprising finding that MIR21, pri-miR-21 and pre-miR-21 each have characteristics that can be exploited to deliver exogenous nucleotide sequences into exosomes.

In part, the invention is based on the surprising finding that the gene MIR21 contains regulatory sequences in the 5′ and 3′ flanking regions of miR-21 that can be exploited to control miRNA expression, and that can also be exploited to deliver exogenous nucleotide sequences into exosomes. In particular, the inventors have found that the MIR21 promoter can be regulated in a cell-specific and/or stimulus-specific manner, for example by transcriptional activation, use of other microRNAs, anti-miRs or any other technology currently employed in recombinant gene engineering. This regulation may be by a mechanism (or mechanisms) that involves the expression of the transcription factor c-Myc, as demonstrated in the Examples. The inventors have also identified a region in the 5′ upstream sequence to miR21 that acts as a repressor of the expression of miR21. Controlling the function (e.g. presence or absence) of the identified regulatory sequences can therefore be used to control the delivery of exogenous nucleotide sequences into exosomes.

One or more of the regulatory elements located in the upstream and downstream region of the MIR21 gene can therefore be used to control miRNA loading into exosomes. Any miRNA (or other nucleic acid of interest) can be loaded and controlled by engineering the desired regulatory regions. In some embodiments, the regulatory element comprises all or part of the miPPR-21 promoter region that is found in pri-miR-21 at -3,770 to -3,337 base pairs upstream to the miR-21 hairpin. In some embodiments, the regulatory element comprises a repressor from pri-miR-21. In some embodiments, the repressor from pri-miR-21 is deleted or inactivated. The identification of these miRNA-loading regulatory elements further allows, in some embodiments, to drive miRNA loading into exosomes by contacting the producer cell containing those elements with a transcription factor(s) or other agent(s) that bind to the regulatory elements. In certain embodiments, site-directed genome editing (e.g. CRISPR) can be used to add or insert a desired exogenous RNA, for example mature miRNA, sequence to the MIR21 locus, and therefore utilise the MIR21 control and exosome-loading mechanism for any desired miRNA sequence (or other nucleic acid cargo). This can then be expressed in a cell ex vivo or in vivo. In further embodiments the construct containing the pri and/or pre-miR-21-5p regulatory elements alongside an RNA cargo may be engineered to include one or more non-natural nucleotides, which can be synthesised ex-vivo and transfected into cells or used as a therapeutic or gene delivery vehicle in vivo.

Furthermore, the inventors consider that expression of an engineered RNA including exogenous RNA cargo can be regulated by a number of identified transcription factors, which can impart a degree of cell-specific expression whereby cells expressing such identified transcription factors will transcribe more of the cargo RNA than cells that do not express such transcription factors. This may be of interest in certain disease states such as cancer where these transcription factors can be upregulated.

Moreover, the inventors have identified that there are several wobble pairs, mismatches and deletions contained within the pre-miR-21 stem and loop sequences that are important for exosomal packaging, and that can be exploited to deliver exogenous nucleotide sequences into exosomes. These exosomes may have subsequent therapeutic use or be used in vitro as a delivery system for recombinant or exogenous RNA into a recipient or target cell. The inventors have yet further identified a region in the 3′ downstream sequence of miR21 that acts as a stabilization sequence for the correct expression of MIR21. Without being bound by theory, these structural features of miR-21-5p are thought to be important for the correct processing, spatial folding, and sorting of these small non-coding nucleotides into exosomes.

Accordingly, a nucleotide sequence of interest can be inserted into the pri-miRNA-21 or the pre-miR-21 sequence, in the place of the native mature miR-21 miRNA sequence, and can be expected to be delivered into exosomes.

The invention therefore provides a nucleic acid construct, based on the primary, secondary and/or tertiary structure of pri-miR-21 or pre-miR-21 that is modified to contain an exogenous nucleotide sequence. This construct is useful for being selectively loaded into exosomes. This construct is particularly useful for loading ribonucleic acid cargoes, such as microRNAs, antimiRs, morpholinos, antisense oligonucleotides, shRNA (short hairpin RNA) and small mRNAs into exosomes. Such genetic manipulation can be achieved by gene editing techniques using site specific endonucleases such as zinc finger endonucleases, CRISPR/Cas, TALENs and prime editing commonly known in the art.

In certain embodiments, the modified pri- or pre-miR-21 comprises one or more of: a promoter region; a repressor; an enhancer; and/or a stabilizer of the expression of the RNA for miR21-5p; typically to allow the expression of exogenous nucleic acid (e.g. miRNA) in any cell by a controlled mechanism to drive the correct processing of the exogenous nucleic acid (e.g. miRNA).

In some embodiments, the modified sequence also comprises wobble pairs, which are thought to distort the RNA deep groove to allow recognition of the pre-miRNA by RNA binding Proteins (RBPs). These RBPs typically have high affinity for the ceramides located in the exosomal membrane. Therefore, by retaining the structural and functional features of the native pri and/or pre-miR-21 a nucleotide sequence of interest can be targeted into exosomes by a controlled expression mechanism.

A first aspect of the invention provides a pre-miRNA for targeting an exogenous nucleotide sequence to an exosome, wherein the pre-miRNA comprises a stem loop structure, and wherein the stem comprises at least one wobble pair.

Another aspect of the invention provides a pri-miRNA for the expression of a pre-miRNA for targeting an exogenous nucleotide sequence to an exosome. The pri-miRNA expression typically is driven by a promoter region, and contains regulatory and stabilizing sequences, wherein the pre-miRNA comprises a stem loop structure, and wherein the stem comprises at least one wobble pair. Typically, the expression is controllable and/or at a high level.

Another aspect of the invention provides MIR21 as a locus that can be genetically modified to replace the natural sequence of the pre-miR-21 by an exogenous nucleotide sequence to target that exogenous sequence to an exosome. Such genetic manipulation can be achieved by gene editing techniques using site specific endonucleases such as zinc finger endonucleases, CRISPR/Cas, TALENs and prime editing commonly known in the art.

The modified pri- and/or pre-miR-21 may also include mismatches and deletions, which are also thought to assist in determining the three-dimensional structure of the pre-miRNA and therefore facilitate recognition by RBPs. In certain embodiments, the pre-miRNA of the invention further comprises: a pre-miRNA 5′ end comprising a wobble pair; and/or an exogenous nucleotide sequence comprising one or more deletions and/or mismatches; and/or a pre-miRNA 3′ end comprising a loop and wobble pair.

In some embodiments, the pre-miRNA may comprise a stem-loop secondary structure having one or more wobble pairs in the stem. In an embodiment of the invention, the wobble pairs may be from 1 to 5 nucleotides in length, for example 1, 2, 3, 4 or 5 nucleotides in length.

In another embodiment, the pre-miRNA may comprise a stem-loop secondary structure comprising one or more base pair mismatch(es) in the stem. These mismatched base pairs give rise to unpaired regions within the stem. In some embodiments, mismatched regions may be from 1 to 5 nucleotides in length, for example 1, 2, 3, 4, or 5 nucleotides in length. A mismatch is shown in the position marked by a hash (#) in FIG. 5B.

In another embodiment, the pre-miRNA may comprise a stem-loop secondary structure comprising one or more base deletions in the stem. In some embodiments, the deletions may be from 1 to 5 nucleotides in length, for example 1, 2, 3, 4, or 5 nucleotides in length. Typically, the deletion leaves one or more nucleotides in the stem region without a corresponding base opposite it in the stem (i.e. so the two halves of the stem duplex are different lengths). This can be viewed as an asymmetric stem duplex, for example where one side of the duplex has 20 nucleotides and the other has 22 nucleotides. A deletion is shown in the position marked by an asterisk (*) in FIG. 5B.

Any suitable exogenous nucleotide sequence can be employed according to the present invention. This sequence is exogenous because it replaces all or part of the native mature miR-21 in pre-miR-21. Typically the exogenous sequence is RNA. It may be on oligonucleotide or a polynucleotide. It is typically single-stranded RNA. In some embodiments, the exogenous nucleotide sequence is RNA, miRNA, shRNA, sgRNA or guide RNA for use in gene editing. In some embodiments the exogenous nucleotide sequence in the pre-miRNA of the invention is 19-25 nucleotides in length. In a further embodiment, the length of the exogenous nucleotide sequence is 19, 20, 21, 22, 23, 24, or 25 nucleotides in length. In a further embodiment of the invention, the exogenous nucleotide sequence is a nucleotide sequence for a shRNA, siRNA, miRNA, anti-miR, antisense oligonucleotide (ASO), CRISPR guide RNA, or any other exogenous nucleotide sequence. In an exemplary embodiment, the exogenous nucleotide sequence is a mature miR that is not mature miR-21, and optionally comprises a duplex comprising at least one strand consisting of 21, 22 or 23 nucleotides. In one embodiment the exogenous sequence has the 20/22 asymmetric nucleotide structure of miR-21, as shown in FIG. 5B.

In an embodiment, the pre-miRNA comprises: wobble pairs from 1 to 5 nucleotides in length, for example 1, 2, 3, 4 or 5 nucleotides in length; and/or mismatches from 1 to 5 nucleotides in length, for example 1, 2, 3, 4, or 5 nucleotides in length; and/or deletions from 1 to 5 nucleotides in length, for example 1, 2, 3, 4, or 5 nucleotides in length; and/or an exogenous nucleotide from 19, 20, 21, 22, 23, 24, or 25 nucleotides in length.

In certain embodiments, the pre-miRNA comprises a stem-loop secondary structure comprising an overall loop length of 4, 5, 6, 7, 8 or 9 nucleotides. In one embodiment, the loop is 7 nucleotides in length. In a further embodiment the loop structure comprises or consists of the sequence 5′-CUCUAAG-3′. In certain embodiments, the pre-miRNA may comprise a stem-loop secondary structure comprising an overall stem length of 25 to 35 nucleotides, for example 25, 26, 27, 28, 29, 30, 31, 32, 33, 34 or 35 nucleotides.

The structure of the pre-miRNA is considered to be important for its exosome packaging. Therefore, in some embodiments, the modified pre-miR-21 is structurally similar to the primary, secondary, and/or tertiary structure of the native miR21-5p.

In a certain embodiment of the invention, the pre-miRNA comprises the structure A-B-C, wherein A is a pre-miRNA 5′ end, wherein the pre-miRNA 5′ end comprises at least 50%, 60%, 70%, 80%, 90%, or 100% sequence identity to the 5′ end of pre-miR-21; B is an exogenous nucleotide sequence not naturally found in pre-miR-21; and C is a pre-miRNA 3′ end, wherein the pre-miRNA 3′ end comprises at least 50%, 60%, 70%, 80%, 90%, or 100% sequence identity to the 3′ end of pre-miR-21, optionally wherein the total pre-miRNA comprises at least 20%, 30%, 40%, 50%, 60%, 70% or greater total sequence identity to the total sequence of pre-miR-21.

By “5′ end” it is meant the sequences on both strands of the stem duplex at the non-loop (open) end, adjacent to the mature miRNA/exogenous sequence. This is depicted in FIG. 5B. Typically, the 5′ end-sequence comprises 7 to 9 nucleotides on each strand of the duplex. Typically, the 5′ end sequence is 8 nucleotides in length on each side of the pre-miRNA duplex.

By “3′ end” it is meant the end of the duplex that comprises the loop and sits adjacent to the mature miRNA/exogenous sequence. The 3′-end comprises the loop structure and at least one or two wobble pairs. The 3′-end is labelled as the 3′-loop in FIG. 5B.

In a particular embodiment of the invention, the pre-miR-21 structural mimic comprises:

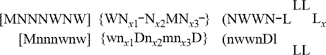

wherein:
N is any nucleotide and hybridises with n, optionally by Watson-Crick pairing;
Nₓ is any length of nucleotide sequence that hybridises with n, optionally wherein Nₓ₁,
Nₓ₂, and Nₓ₃ have different lengths;
M is a nucleotide and is mismatched or does not hybridise with m;
D is a nucleotide and is present on one side of the stem loop structure only;
W is a nucleotide and forms a wobble pair with w;
L is a nucleotide that forms a loop structure and may hybridise with I; wherein Lₓ may be from 1-5 nucleotides
[...] is A, a miR-21 5′ end structural mimic;
{...} is B, an exogenous nucleotide sequence; and
(...) is C, a miR-21 3′ end structural mimic.

In some embodiments, Lₓ comprises 4, 5, 6, 7, 8 or 9 nucleotides. In one embodiment, the loop is 7 nucleotides in length. In a further embodiment the loop structure comprises or consists of the sequence 5′-CUCUAAG-3′.

In a certain embodiment of the invention, the pre-miRNA has the structure:

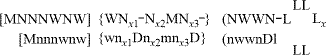

wherein:
N is any nucleotide and hybridises with n, optionally by Watson-Crick pairing
Nₓ is any length of nucleotide sequence that hybridises with n, optionally wherein Nₓ₁,
Nₓ₂, and Nₓ₃ have different lengths;
M is a nucleotide and is mismatched or does not hybridise with m;
D is a nucleotide and is present on one side of the stem loop structure only;
W is a nucleotide and forms a wobble pair with w;
L is a nucleotide that forms a loop structure and may hybridise with I; and
wherein the pre-miRNA comprises the structure A-B-C, wherein:
   [...] is A, a miR-21 5′ end structural mimic;
   {...} is B, an exogenous nucleotide sequence; and
   (...) is C, a miR-21 3′ end structural mimic.

Any suitable exogenous nucleotide sequence (Nₓ) can be employed according to the present invention. In some embodiments, Nₓ is 1, 2, 3, 4, 5 or 6 nucleotides in length. In certain embodiments, Nₓ₁ is 1, 2, 3, 4, 5, 6, 7, 8, 9, 10, 11, 12, 13, 14, 15, 16, 17, or 18 nucleotides in length; and/or Nₓ₂ is 1, 2, 3, 4, 5, 6, 7, 8, 9, 10, 11, 12, 13, 14, 15, 16, 17, or 18 nucleotides in length; and/or Nₓ₃ is 1, 2, 3, 4, 5, 6, 7, 8, 9, 10, 11, 12, 13, 14, 15, 16, 17, or 18 nucleotides in length; or any combination thereof. In some embodiments, Nₓ₁ comprises from 4 to 12 nucleotides; and/or Nₓ₂ comprises from 2 to 8 nucleotides; and/or Nₓ₃ comprises from 2 to 8 nucleotides, or any combination thereof. Typically, N_{X1} is 7, 8, or 9 nucleotides in length, and/or Nₓ₂ is 4, 5, or 6 nucleotides in length, and/or N_{X3} is 4, 5, or 6 nucleotides in length, or any combination thereof. In one embodiment, N_{X1} is 8 nucleotides in length, Nₓ₂ is 5 nucleotides in length, and N_{X1} is 5 nucleotides in length.

In an embodiment of the invention, the pre-miRNA comprises a stem loop secondary structure having an overall length of about 60 to 80 nucleotides, optionally wherein the exogenous nucleotide sequence is 19-25 nucleotides. In certain embodiments, the stem-loop secondary structure may comprise an overall length of about 60, 65, 70, 75 or 80 nucleotides. Typically, the cleavage site (or sites) required for processing into mature miRNA is retained.

In a certain embodiment, the pre-mRNA comprises the structure:

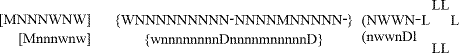

wherein:
N is any nucleotide and hybridises with n, optionally by Watson-Crick pairing;
M is a nucleotide and is mismatched or does not hybridise with m;
D is a nucleotide and is present on one side of the stem loop structure only;
W is a nucleotide and forms a wobble pair with w;
L is a nucleotide that forms a loop structure and may hybridise with I; and
wherein the pre-miRNA comprises the structure A-B-C, wherein:
   [...] is A, a pre-miRNA 5′ end;
   {...} is B, an exogenous nucleotide sequence; and
   (...) is C, a pre-miRNA 3′ end.

In some embodiments, the invention provides a pre-miRNA comprising the sequence of pre-miR-21 that is not the mature miRNA, and an exogenous nucleic acid in place of the mature miRNA.

In some embodiments, the wobble pairs (W-w) comprise guanine-uracil (G-U or U-G), hypoxanthine-uracil (I-U or U-I), hypoxanthine-adenine (I-A or A-I), and/or hypoxanthine-cytosine (I-C or C-I). In some embodiments, the wobble pairs are different. For example, more or more wobble pair may be comprise guanine-uracil (G-U or U-G), hypoxanthine-uracil (I-U or U-I), hypoxanthine-adenine (I-A or A-I), and/or hypoxanthine-cytosine (I-C or C-I), in any order and in any combination. In other embodiments, the wobble pairs are the same, for example all guanine-uracil (G-U or U-G), hypoxanthine-uracil (I-U or U-I), hypoxanthine-adenine (I-A or A-I), or hypoxanthine-cytosine (I-C or C-I). Typically, wherein the wobble pair comprises guanine-uracil (G-U or U-G).

In a certain embodiment, the pre-miRNA comprises the structure: UC

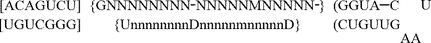

wherein:
N is any nucleotide and hybridises with n, optionally by Watson-Crick pairing;
M is a nucleotide and is mismatched or does not hybridise with m;
D is a nucleotide and is present on one side of the stem loop structure only; and
wherein the pre-miRNA comprises the structure A-B-C, wherein:
   [...] is A, a pre-miRNA 5′ end;
   {...} is B, an exogenous nucleotide sequence; and
   (...) is C, a pre-miRNA 3′ end.

For the avoidance of doubt, the 5′-end, exogenous sequence and 3′-end in this embodiment are as follows:

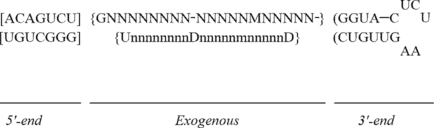

In another embodiment, the pre-miRNA comprises the structure:

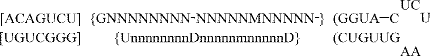

wherein:
N is any nucleotide and hybridises with n, optionally by Watson-Crick pairing;
M is a nucleotide and is mismatched or does not hybridise with m;
D is a nucleotide and is present on one side of the stem loop structure only; and
wherein the pre-miRNA comprises the structure A-B-C, wherein:
   [...] is A, a pre-miRNA 5′ end;
   {...} is B, an exogenous nucleotide sequence; and
   (...) is C, a pre-miRNA 3′ end.

Any suitable exogenous nucleotide sequence can be employed according to the present invention. In a further embodiment of the invention, the exogenous nucleotide sequence is a nucleotide sequence for a siRNA, miRNA, anti-miR, antisense oligonucleotide (ASO), CRISPR guide RNA, or any other exogenous nucleotide sequence. In a further embodiment of the invention, the exogenous nucleotide sequence is a miRNA e.g. miR-146-b or miR-1246, or an shRNA equivalent of any chosen siRNA. Typically, the exogenous nucleotide sequence of the invention is a miRNA.

In some embodiments, the nucleic acid construct comprising the exogenous nucleotide sequence is targeted to the exosome during exosome biogenesis.

In some embodiments, the exogenous nucleotide sequence modulates gene activity in a target cell. In some embodiments, modulating gene activity results in down-regulation of gene expression. In some embodiments, modulating gene activity results in an up-regulation of gene expression. In a further embodiment, the exogenous nucleotide sequence of the invention results in a reduction of gene expression by at least 10%, 20%, 30%, 40%, 50%, 60%, 70%, 80%, 90% or greater. Typically, gene expression is reduced by 60% to 100%. In some embodiments of the invention, the exogenous nucleotide sequence of the invention results in an up-regulation of gene expression. In a further embodiment, the exogenous nucleotide sequence of the invention results in an increase in gene expression by at least 10%, 20%, 30%, 40%, 50%, 60%, 70%, 80%, 90% or greater. Typically, gene expression is increased by 60% to 100%. Modulation of gene activity can be assessed using western blot, qPCR, fluorescence reporter assay, or luciferase reporter assay. Alternatively, modulation of gene activity can be assessed by measuring the therapeutic outcome, typically by ameliorating the symptoms of the disease of interest.

In an embodiment, the pre-miRNA of the invention, sometimes termed a pre-miR-21 structural mimic, retains the biological function of pre-miR-21. A “pre-miR-21 structural mimic” is a pre-miRNA that is substantially structurally and/or functionally similar to pre-miR-21. The pre-miR-21 structural mimic may differ in sequence but retains one or more biological function of pre-miR-21. By “retains one or more biological function” it is meant that the mimic retains at least 95%, 90%, 80% 70%, 60%, 50% of the biological function in question. In a particular embodiment, the biological function in question may be sorting of the pre-miR-21 structural mimic to exosomes. In this embodiment, the pre-miRNA of the invention is an miRNA that may differ in sequence but shares structurally and/or functionally similar, primary, secondary and/or tertiary structure as the native miR21-5p such that it is packaged into exosomes at least 95%, 90%, 80% 70%, 60%, 50% of the level of native pre-miR-21. The amount of exogenous nucleotide sequence present in a purified exosomes can be quantified using qPCR.

In another embodiment, the pre-miRNA of the invention shares structurally and/or functionally similar, primary, secondary and/or tertiary structure such that it is able to bind to RBPs with a binding affinity of at least 95%, 90%, 80% 70%, 60%, 50% of the binding affinity of native pre-miR-21. In another embodiment, the pre-miRNA of the invention shares structurally and/or functionally similar, primary, secondary and/or tertiary structure such that it is able to bind to miRNA processing enzymes with a binding affinity of at least 95%, 90%, 80% 70%, 60%, 50% of the binding affinity of native pre-miR-21. Examples of miRNA processing enzymes include Ran-GTP, Exportin-5, and Dicer. Binding affinity of the pre-miRNA can be measured by, for example, Fluorescence Polarisation (FP), Fluorescence Resonance Energy Transfer (FRET), and Surface Plasmon Resonance (SPR). Other suitable techniques include FISH/SCOPE optionally together with classical IHC/ICC to detect specifically the binding of the miRNA to the RBPs, or to perform an RNA-immunoprecipitation assay (RIP-ChIP assay).

To detect just the presence of the RNA in the exosomes, FISH/SCOPE and/or qPCR is suitable.

In some embodiments, the pre-miRNA comprising an exogenous nucleotide sequence comprises at least 20%, 25%, 30%, 35%, 40%, or greater sequence identity to the sequence of native pre-miR-21-5p. The engineered pre-miRNA is intended to mimic the three-dimensional structure of the native pre-miR-21, such that the pre-miRNA of the invention is targeted into the exosomes during exosome biogenesis like the native pre-miR-21. This is termed a pre-miR-21 structural mimic.

In a certain embodiment, the exogenous nucleotide sequence may retain the native primary, secondary and/or tertiary structure of the stem of mature miR21-5p, with respect to length, wobble pairs, mismatches, and deletions of the pre-miR-21-5p. In another embodiment, the exogenous nucleotide sequence comprises the following features that are present in the native structure of the stem of mature miR-21-5p: the overall length of miR-21-5p; and/or wobble pairs; and/or mismatches; and/or deletions. In some embodiments, the exogenous nucleotide sequence is longer or shorter than miR-21-5p, and/or the exogenous nucleotide sequence comprises an increased or decreased number of wobble pairs compared to miR-21-5p, and/or the exogenous nucleotide sequence comprises an increased or decreased number of mismatched base pairs compared to miR-21-5p, and/or the exogenous nucleotide sequence comprises an increased or decreased number of deletions compared to miR-21-5p.

In a second aspect of the invention, a cassette comprising the pre-miRNA of the invention is provided. In some embodiments, the cassette comprises: 5′ pri_miR-21 sequence; a pre-miRNA according to any preceding claim; and a 3′ pri_miR-21 sequence (in that order).

In certain embodiments, the miRNA expression cassette preserves the original sequences and length of the pri- and pre-miRNA including the loop, miss-pairs, Wobble pairs and lack of base-pairing of within the native pre-miR-21.

In an exemplary embodiment, the cassette comprises: a ubiquitous promoter region; a cell specific promoter or a promoter with specific and/or selected transcription factor binding sites; an enhancer or repressor sequence in the 5′ upstream sequence of the miRNA; and/or a 3′ enhancer, repressor or stabilizing sequence in the 3′ downstream sequence of the miRNA.

In some embodiments, the native miR-21 repressor sequence is not present or is not functional.

In one embodiment of the invention, the inventors have designed and generated a miRNA expression cassette that preserves the original sequences and length of the pri- and pre-miRNA including the loop, mismatched-pairs, wobble pairs and lack of base-pairing within the original naturally occurring miRNA. This is intended to maintain the structure generated by the mimic that makes use of the pre-miR-21 exosomal shuttling sequences and function.

A third aspect of the invention provides a vector comprising a cassette of the invention or a pre-miRNA of the invention. In some embodiments, the vector of the invention comprises adenoviral vectors, adeno-associated viral vectors, the pEF1-alpha, pTK, pCAG, pSV and the pCMV series of plasmid vectors, vaccinia, and retroviral vectors, as well as baculovirus. In a particular embodiment, the vector of in the invention comprises a lentiviral vector. In some embodiments the vector comprises the promoters for cytomegalovirus (CMV), CAG, EF1-alpha, TK and SV40.

A fourth aspect of the invention provides a cell comprising a vector, cassette, pre-miRNA, or CRISPR/Cas9 or genetically modified locus of the disclosure. The cell that can be used is not particularly limited, and the skilled person will be aware that a wide range of cells can be used. In some embodiments, the cell is a stem cell or a dendritic cell. In some embodiments, the cell is a stem cell, optionally a neural stem cell or a mesenchymal cell. In a particular embodiment the cell is a neural stem cell. In a further embodiment, the cell is a CTX0E03 cell (deposited by the applicant at the ECACC with Accession No. 04091601). In another embodiment, the cell is optionally a partially differentiated stem cell. The vector can be introduced into the cell by any known method of introducing a vector or nucleic acid into the cell. Such methods may include but are not limited to transfection using a cationic lipid reagent, electroporation, and viral transduction.

A fifth aspect provides a method of loading exosomes with an exogenous nucleotide sequence comprising producing exosomes from the cell comprising the construct of the invention. In particular embodiments, the invention enables the loading of any gene silencing or modifying oligonucleotides including an shRNA coding for the intracellular production of a specific siRNA, or an siRNA, miRNA, or antisense oligonucleotide (ASO), or a morpholino or sgRNA or guide RNA for gene editing. In further embodiments, the invention enables the loading of any gene editing tool such as a CRISPR RNA guide strand, and any other single stranded RNA molecule. In a particular embodiment, the nucleotide sequence comprises or consists of a miRNA nucleotide sequence. By “loaded” it is meant into the exosome, on the surface of the exosome, and in the membrane of the exosome. Loading into the exosome may be loading inside the exosome, i.e. into the lumen.

In a further aspect of the invention, a method of preparing exosomes comprising the pre-miRNA or mature miRNA of the invention is provided. The method of preparing exosomes comprising the pre-miRNA or mature miRNA comprises: culturing cells and harvesting of conditioned media. In an embodiment, the method further comprises the optional steps of purification of the exosomes and validation of the exosomes. In a further embodiment, the method comprises culturing the cells, harvesting of conditioned media, and optionally purification, and optionally validation of the exosomes. In some embodiments, an exosome that is obtained or obtainable from the method is provided. As will be understood, exosomes or exosome-like vesicles may be purified by any method known in the art.

A seventh aspect of the invention provides an exosome comprising a pre-miRNA of the invention. In a further embodiment of the invention, the pre-miRNA comprises an exogenous nucleotide sequence comprising a nucleotide sequence for an shRNA, siRNA, miRNA, anti-miR, antisense oligonucleotide (ASO), CRISPR guide RNA, or other exogenous nucleotide sequence. In some embodiments, the exogenous nucleotide sequence could be a miRNA e.g. miR-146-b or miR-1246, or an shRNA coding for the intracellular production of a specific siRNA. Typically, the exogenous nucleotide sequence of the invention is an miRNA.

A further aspect of the invention provides a method of delivering an exogenous nucleotide sequence to a target cell, using an exosome loaded with mature miRNA produced according to the invention. In an embodiment, the method comprises contacting the target cell with the miRNA loaded exosomes. In a further embodiment, the method optionally comprises first, a step comprising isolating the exosomes loaded with miRNA via the pre-miRNA, and second, a step comprising contacting the target cell with the miRNA loaded exosomes. In some embodiments, the contacting the target cell with the miRNA loaded exosomes step is for at least 1 hour, 2 hours, 3 hours, 4 hours, 5 hours, 24 hours, or greater. In another embodiment the contacting step occurs at 37° C.

A ninth aspect of the invention provides a method of modulating gene activity in a target cell, comprising administering an exosome comprising RNA, generated according to the invention. In some embodiments, modulating gene activity results in down-regulation of gene expression. In some embodiments, modulating gene activity results in an up-regulation of gene expression. In some embodiments of the invention, the exogenous nucleotide sequence of the invention results in a down-regulation of gene expression. In a further embodiment, the exogenous nucleotide sequence of the invention results in a reduction of gene expression by at least 10%, 20%, 30%, 40%, 50%, 60%, 70%, 80%, 90% or greater. Typically, gene expression is reduced by 60% to 100%. In some embodiments of the invention, the exogenous nucleotide sequence of the invention results in an up-regulation of gene expression. In a further embodiment, the exogenous nucleotide sequence of the invention results in an increase in gene expression by at least 10%, 20%, 30%, 40%, 50%, 60%, 70%, 80%, 90% or greater. Typically, gene expression is increased by 60% to 100%. Modulation of gene activity can be assessed using western blot, qPCR, fluorescence reporter assay, or luciferase reporter assay. Alternatively, modulation of gene activity can be assessed by measuring the therapeutic outcome, typically by ameliorating the symptoms of the disease of interest.

A further aspect of the invention provides a pharmaceutical composition comprising the loaded exosome of the invention. A pharmaceutically acceptable composition typically includes at least one pharmaceutically acceptable carrier, diluent, vehicle and/or excipient in addition to the exosomes of the invention.

Another aspect of the invention provides an exosome comprising the RNA cargo generated according to the methods of the invention for use in therapy. The therapy may be of a disease requiring inhibition of cell migration, such as cancer, fibrosis, atherosclerosis or rheumatoid arthritis. The therapy may be of a neurological disease, an ophthalmic disease, hearing loss, inflammation, cancer, or viral infection. The therapy may also be of a disease requiring inhibition of angiogenesis, such as treating a solid tumour by inhibiting angiogenesis. In some embodiments, the invention provides a miRNA loaded exosome for use in gene therapy. In further embodiments, the invention provides gene therapy by editing point mutations that cause diseases, inactivating (“knocking out”) a mutated gene that is functioning improperly, and/or introducing a new gene into the body to help fight a disease. Examples of diseases that can be treated by gene therapy include, but are not limited to, cystic fibrosis, severe combined immune deficiency (ADA-SCID), chronic granulomatous disorder (CGD), haemophilia, Leber’s congenital amaurosis (LCA), cancers, Parkinson’s disease and Huntington’s disease. In some embodiments, the gene therapy is mediated by the pre-miRNA of the invention comprising guide RNA, and CRISPR genome editing.

### BRIEF DESCRIPTION OF THE DRAWINGS

FIG. 1: A new plasmid containing the gene MIR21. This figure shows that a new plasmid containing the gene MIR21 (shown in FIG. 1A) is active in two different cells (HEK and CTX see FIGS. 1B to 1D) despite in HEK cells the basal expression level being extremely low.

FIG. 2: The MIR21 plasmid contains different regulatory sequences. This figure shows that the MIR21 plasmid contains different regulatory sequences in the promoter region that can be activated by PMA (phorbol 12-myristate 13-acetate) and c-myc. It also shows that there are other sequences in the 5′ region that contains at least a repressor sequence, and a stabilising sequence in the 3′ region that also controls the levels of the mRNA for miRNA.

FIG. 3: Example of construction of hybrid cassette. The inventors have shown that it can be important to have recognition that relies on the 3D structure of the loaded miRNAs. The 3D structure depends on the internal loops present in the miRNAs.

FIG. 4: Exosomes as vehicles for miRNAs: miRNAs based on miR-21-5p cassette and structure. Exosomes are natural vehicles for miRNAs. The inventors have modified the sequence for miR21-5p in order to carry a designed miRNA against the fluorescent protein Ruby2. This can be used to introduce any miRNA into exosomes. A) miR-21-5p cassette and structure, B) miR-21 structural mimic loaded into an exosome.

FIG. 5: Design of a miR-21-based pri-miRNA cassette for the expression and loading of therapeutic RNAs in Exosomes. (A) Wobble pairs confer a different three-dimension structure to the RNA helix (Adapted from Varani and McClain, EMBO Reports, 2000). (B) Native structure of the pre-miR21-5p containing several Wobble G-U pairs (coloured boxes), deletions (*) and mismatch (#) that are maintained in the sequence of the designed pre-miR-Ruby2 to preserve its recognition and processing by RBPs. (C) Structure of the pri-miR-Ruby2 cassette and (D) its position on a modified lentivirus vector containing the promoters EF-1 alpha and CAG, the selection gene for Blasticidin (BSD) and the reporter gene Clover. The pri-miR-Ruby2 cassette was cloned in the vector as a 3′UTR sequence of a coding sequence.

FIG. 6: Functional miR-Ruby2 is loaded into Exosomes of a producer cell line. (A) Expression levels of mature miR-Ruby2 by qPCR on cells electroporated with a vector containing the pri-miR-Ruby2 cassette. (B) HEK293-Ruby2 cells electroporated with a control plasmid expressing Clover (*) or the plasmid containing the pri-miR-Ruby2 cassette and the fluorescent protein Clover (arrowheads). (C) Levels of miR-Ruby2 loaded in Exosomes from HEK293-pri-miR-Ruby2 cells compared to a control cell line determined by qPCR. (D) Expression levels of the protein and (E) mRNA for Ruby2 in a co-culture assay of HEK293-Ruby2 and control cells, or with HEK293 expressing miR-Ruby2.

### DETAILED DESCRIPTION OF THE INVENTION

The present inventors have highlighted a particular pre-miRNA that has particular functions in exosome loading. In particular, the inventors have surprisingly identified that within the sequence of the MIR21 gene there are several regulatory sequences that are important for the expression of the mature miR21-5p. Regulatory sequences are identified in both the pre-and pri-MIR21 regions. Also, within the surrounding sequence of miR-21-5p there are several wobble pairs, mismatches and deletions. These features are contained within the pre-miRNA stem and loop sequences and have been identified as important for exosome packaging. The structural features of miR-21-5p have been found to be important for the correct processing, spatial folding and sorting of these small non-coding nucleotides into exosomes. It has further been identified that the correct processing, spatial folding and sorting of these small non-coding nucleotides into exosomes can all occur in the presence or absence of Ago and Dicer.

The invention therefore provides the use of one or more of the regulatory elements located in the upstream and downstream region of the MIR21 gene to control loading into exosomes, for example loading of ssRNA in particular miRNA.

The invention therefore also provides a pre-miRNA scaffold for targeting an exogenous nucleotide sequence into exosomes. By adding an exogenous oligonucleotide or polynucleotide of interest to the identified scaffold, the scaffold of the invention has the potential to load any exogenous nucleotide sequence of interest (such as miRNA, shRNA, siRNA, anti-miR, ASO, or CRISPR guide strand) into exosomes by genetically modifying exosome producer cells. The invention also provides a pri-miRNA cassette, which has the advantage of not requiring direct tagging of the miRNA with an exosome targeting motif for exosome loading, as described in the prior art. This cassette can be modified to regulate the expression of the exogenous nucleotide sequence (e.g. RNA) depending on the cell line or in order to temporarily control its expression. Also, the invention provides the capacity to modify the endogenous chromosomal locus of MIR21, e.g. using gene editing, in order to replace the pre-miR-21 or a portion thereof by other nucleic sequence maintaining its expression and loading into exosomes.

Moreover, the invention does not necessarily require use of cell lines that lack any of the protein machinery involved in the canonical processing of pre-miRNAs. Without being bound by theory, it is understood that by maintaining the three-dimensional structure of pre-miR-21, the pre-miRNA of the invention is favoured for recognition by RBPs, resulting in the processing, production and loading of the exogenous nucleotide sequence into exosomes.

The invention provides methods for the modification of any producer cell to generate exosomes that are loaded with any nucleotide sequence of interest. The purified exosome of the invention can then be used systemically or locally in vivo for use in therapy. The exosome containing the nucleotide sequence of interest offers improved biodistribution, systemic stability, and improved target tissue uptake compared to naked exogenous nucleotide sequences.

Methods for loading pre-purified isolated exosomes directly with exogenous nucleotide sequences are known in the art. However, these methods have been shown to be highly inefficient, not yet reproducible with unmodified siRNAs, and difficult to scale up using the current methods for loading (e.g. electroporation and lipofection). The present inventors have addressed this problem by modifying a producer cell line with an expression vector encoding a pre-miRNA that targets an exogenous nucleotide sequence of interest into exosomes. The inventors have shown that the subsequently harvested exosomes contain the desired exogenous nucleotide sequence (Example 1).

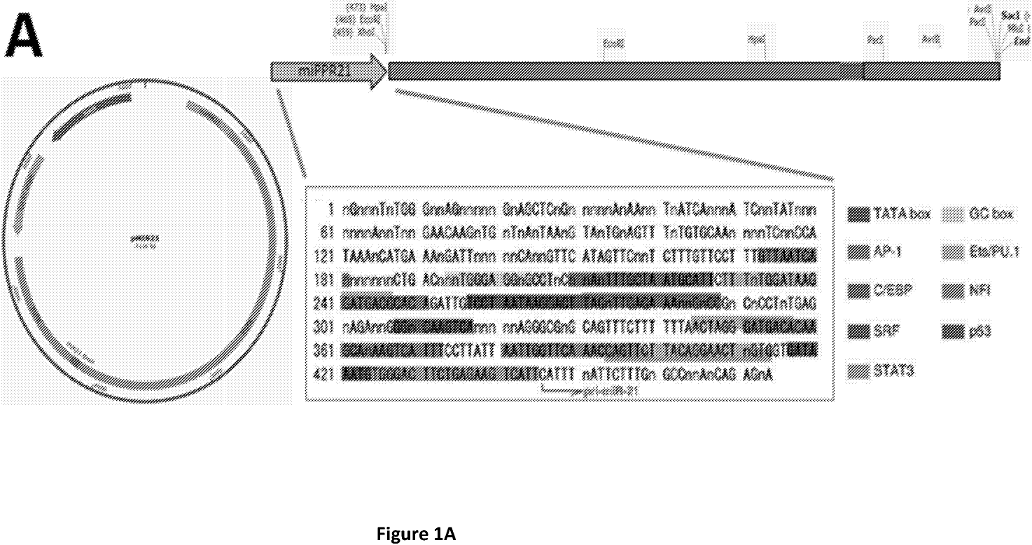

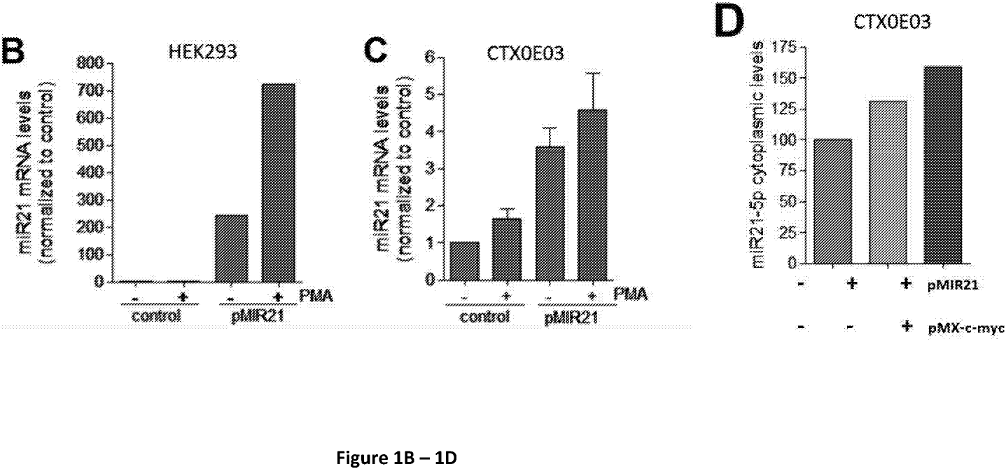

The Examples (e.g. FIG. 1) show that a construct produced according to the invention is active in different cell types. The invention is therefore expected to be broadly applicable across cell types.

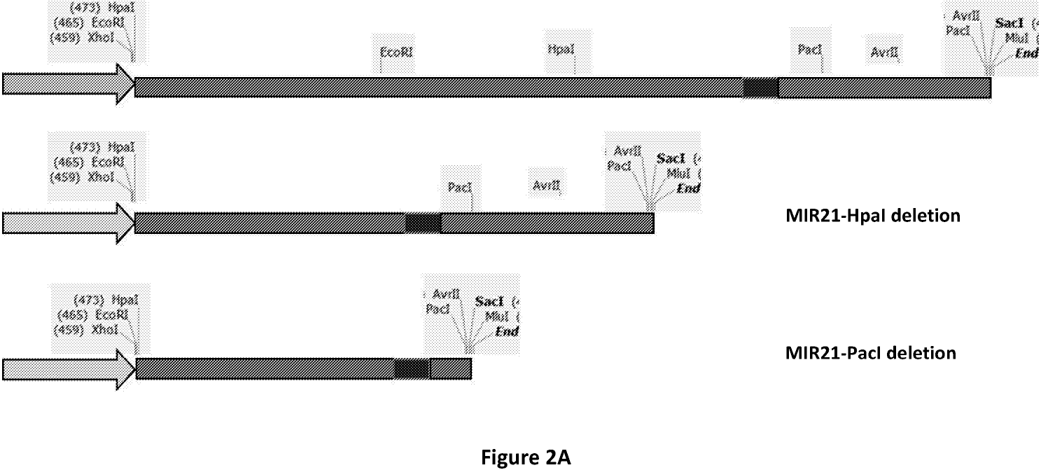

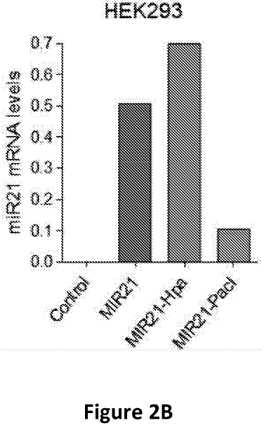

The different regulatory sequences present in MIR21 can be seen in FIG. 2. This Figure shows that the MIR21 plasmid contains different regulatory sequences in the promoter region that can be activated by PMA (phorbol 12-myristate 13-acetate) and c-myc. It also shows that there are other sequences in the 5′ region that contains at least a repressor sequence, and a stabilising sequence in the 3′ region that also controls the levels of the mRNA for miRNA. In particular, it can be seen in FIG. 2 that the miRNA level increases notably when MIR21 is cleaved at the Hpal restriction site, and decreased when cleaved at the Pacl restriction site.

The inventors have identified that sequences within the pri- and pre-miRNA surrounding the mature miRNA sequence can be used to load exosomes with exogenous nucleotides sequences. By assessing the relevant sequences in the functional tertiary loop structure, the inventors have generated a hybrid ‘cassette’ vector, which can be used to load any desired exogenous nucleotide sequence in to an exosome. This hybrid cassette vector typically contains 5′ upstream and 3′ downstream sequences to the miRNA or nucleotide of interest that allows its expression and processing for loading that nucleic acid in exosomes. Such exosomes can be subsequently harvested and used as delivery vehicles containing the desired exogenous nucleotide sequence. To target the loaded exosome to different tissues, the producer cell line can be varied, and/or the surface markers on the exosome can be manipulated.

In some embodiments, exosomes are loaded with RNA cargoes containing modified non-natural nucleotides. The construct of the invention may be synthesised with non-natural nucleotides (substituted for native nucleotides) within the cargo RNA and the resultant mimetic transfected directly in to a target cell. The target cell can then be cultured and the exosomes it produces harvested for therapeutic or diagnostic use or for use in research, for instance to engineer knock-in and knockdown cell lines for the purpose of modelling disease or drug screening.

### MIR-21 and Exosome Loading

miRNAs are short non-coding RNAs (ncRNAs) of around 22 nucleotides that mediate gene silencing by guiding Argonaute (Ago) proteins to target sites in the 3′ untranslated region (UTR) of mRNAs. The miRNA-loaded Ago forms the part of the miRNA-induced silencing complex (miRISC), which promotes translation repression and degradation of targeted mRNAs. The interaction of miRNAs with their targets is largely based on their seed sequence, and miRNA biogenesis is affected by RNA secondary structures that mediate interactions with RBPs. It has been suggested that exosomal RBPs can direct miRNA into exosomes (Gebert, and MacRae. 2019).

Pre-miRNAs are RNA hairpins, around 70 bases long, generated in the nucleus starting from pri-miRNAs after cleavage with Drosha. Pre-miRNAs are exported into the cytoplasm by exportin 5, where they are further processed by the nuclease Dicer to form mature miRNA (Cullen. 2004).

The present inventors have reported that miR-21-5p sequence is an abundantly expressed exosomal miRNA produced by neural-stem cell line CTX0E03 due to its special characteristics and genetic modification by over-expression of the hybrid transcription factor c-myc-ERTam. miR-21, also known as hsa-miR-21, miR-21-5p and hsa-miR-21-5p, is a mammalian miRNA that is encoded by the MIR21 gene. MIR21 is located inside an intronic sequence of the gene VMP1, but expression does not depend on the expression or regulatory sequences of this gene. Within the pri-miR-21 sequence there are several regulatory elements that comprise a promoter region, enhancers, repressors and stabilizing sequences that allows to control and regulate the expression of miR-21-5p. Within the pre-miR-21-5p sequence, there are several key G-U pairs and loop sequences that are essential to its exosomal packaging. It was observed that consistently within the harvested exosomes derived from CTX0E03 that miR21-5p was an abundantly expressed miRNA. Across numerous batches, the abundance of miR-21-5p was approximately at least 1 order of magnitude higher than the next most abundant miRNA. This indicates that there is a strong exosomal packaging function associated with this specific miRNA.

hsa-mir-21 (pre-miR) has the miRBase accession number MI0000077:

>hsa-mir-21 MI0000077UGUCGGGUAGCUUAUCAGACUGAUGUU
GACUGUUGAAUCUCAUGGCAACACCAGUCGAUGGGCUGUCUGACA

The stem-loop structure as depicted on miRBase is:

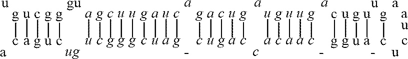

The mature -5p sequence is nucleotides 8 to 29, i.e:

>hsa-miR-21-5p MIMAT0000076UAGCUUAUCAGACUGAUGUUGA

The mature -3p sequence is nucleotides 46 to 66, i.e:

>hsa-miR-21-3p MIMAT0004494CAACACCAGUCGAUGGGCUGU

The invention therefore provides a pre-miRNA, based on the structure of pre-miR-21, modified to contain an exogenous nucleotide sequence for loading into exosomes. This modified pre-miRNA containing an exogenous nucleotide sequence can be described as a pre-miR-21 structural mimic or mimetic. In some embodiments, the modified pre-miR-21 is structurally similar to the primary, secondary and/or tertiary structure of the native pre-miR21-5p. The invention therefore provides a pre-miRNA for targeting an exogenous nucleotide cargo to exosomes, wherein the pre-miRNA comprises a stem loop structure, and wherein the stem comprises a wobble pair.

An “exogenous nucleotide sequence” is a nucleotide sequence that is not naturally found in pre-miR-21-5p. In some embodiments the exogenous nucleotide sequence that is incorporated into the pre-miRNA of the invention is a duplex. In a further embodiment, the exogenous nucleotide sequence is modified to comprise wobble pairs, deletions and/or mismatches. In a particular embodiment, the exogenous nucleotide sequence comprises a mismatch and a deletion in the same positions as the native pre-miR-21-5p sequence, such that it maintains substantially the same three-dimensional structure as the native pre-miR-21-5p. Without being bound by theory, the three-dimensional structure of the pre-mi-21 is thought to be important for its targeting to the exosome during exosome biogenesis. Therefore, a pre-miRNA comprising an exogenous nucleotide sequence that has substantially the same overall three-dimensional structure of pre-miR-21 may target the exogenous nucleotide sequence into the exosome during biogenesis.

A “stem loop” also known as a hairpin loop occurs when two regions of the same strand, usually complementary in nucleotide sequence when read in opposite directions, base-pair to form a double helix that ends in an unpaired loop. A stem loop is a common type of secondary structure in RNA molecules. A stem loop can direct RNA folding, protein structural stability for mRNA, provide recognition sites for RNA binding proteins, and serve as a substrate for enzymatic reactions.

“Nucleic acid hybridisation” occurs when a single-stranded DNA or RNA molecule anneals to complementary DNA or RNA. Hybridisation is a basic property of nucleotide sequences. By “Watson-Crick pairing” it is meant two nucleotides on complementary RNA strands that are connected via hydrogen bonds called a base pair. In Watson-Crick base pairing, adenine (A) forms a base pair with thymine (T) using two hydrogen bonds, and guanine (G) forms a base pair with cytosine (C) using three hydrogen bonds. In canonical Watson-Crick base pairing in RNA, thymidine is replaced by uracil (U).

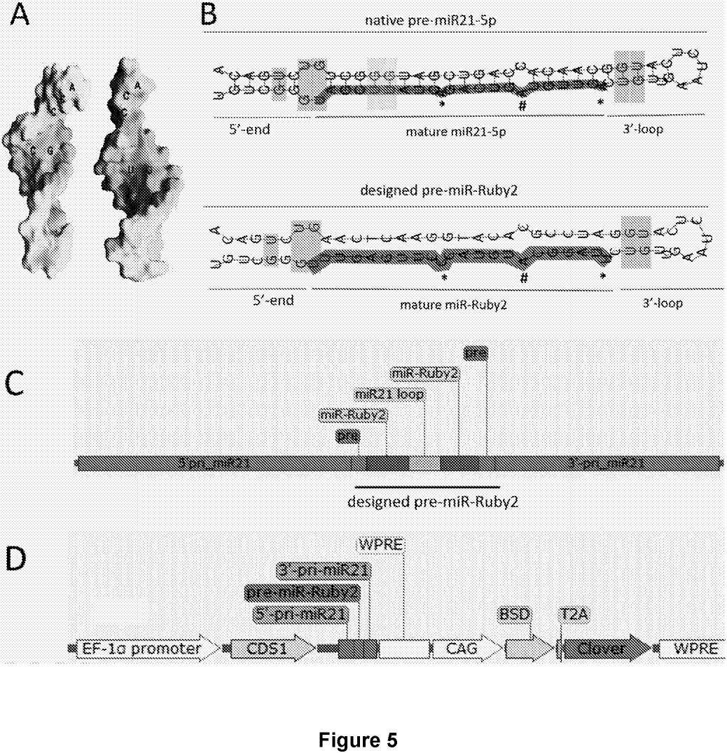

By “wobble pair” it is meant a pairing between two nucleotides in the pre-miR-21 structural mimic that does not follow Watson-Crick base pair rules. In some embodiments, the wobble pairs (W-w) comprise guanine-uracil (G-U or U-G), hypoxanthine-uracil (I-U or U-I), hypoxanthine-adenine (I-A or A-I), and/or hypoxanthine-cytosine (I-C or C-I). In some embodiments, the wobble pairs are different. For example, more or more wobble pair may be comprise guanine-uracil (G-U or U-G), hypoxanthine-uracil (I-U or U-I), hypoxanthine-adenine (I-A or A-I), and/or hypoxanthine-cytosine (I-C or C-I) in any order and in any combination. In other embodiments, the wobble pairs are the same, for example all guanine-uracil (G-U or U-G), hypoxanthine-uracil (I-U or U-I), hypoxanthine-adenine (I-A or A-I), or hypoxanthine-cytosine (I-C or C-I). Typically, the wobble pair comprises guanine-uracil (G-U or U-G). Wobble pairs are fundamental in RNA secondary structure and are critical for the proper translation of genetic code. Their geometric dissimilarity with the Watson-Crick base pairs imparts structural variations decisive for biological functions. In some embodiments, the pre-miRNA of the invention is functionally similar to the native pre-miR-21. Without wishing to be bound by theory, the Wobble pairs are understood to distort the RNA deep groove to allow the native folding of the RNA and its recognition by RBPs, thereby allowing for the processing, production and loading of the miRNA into exosomes (FIG. 5A).

A “mismatch” occurs when two non-complementary bases are aligned in the same base-pair step of a duplex of RNA. Without being bound by theory, it is understood that mismatched base pairs will give the miRNA a different spatial conformation, which will also be recognised by specific RBPs, thereby allowing for the processing, production and loading of the miRNA into exosomes.

By “deletion”, it is meant that a nucleotide is present on one side of the stem loop structure only, with a deletion in the corresponding position on the other side of the stem.

The pre-miRNA of the invention, also termed a pre-miR-21 structural mimic, provides: a pre-miR-21 5′ end structural mimic; an exogenous nucleotide sequence; and a pre-miR-21 3′ loop structural mimic. By “5′ end” it is meant the 5′ end-sequence of 7 to 9 nucleotides on each side of the pre-miRNA duplex. Typically, the 5′ end sequence is 8 nucleotides in length on each side of the pre-miRNA duplex. By “3′ end” it is meant the end of the pre-miRNA duplex that comprises a loop structure.

The pre-miRNA of the invention comprises the structure A-B-C, wherein: A is a pre-miRNA 5′ end, wherein the pre-miRNA 5′ end comprises at least 50%, 55%, 60%, 65%, 70%, 75%, 80%, 85%, 90%, 95% or greater sequence identity to the 5′ end of pre-miR-21; B is an exogenous nucleotide sequence not naturally found in pre-miR-21; and C is a pre-miRNA 3′ end, wherein the pre-miRNA 3′ end comprises at least 50%, 55%, 60%, 65%, 70%, 75%, 80%, 85%, 90%, 95% or greater sequence identity to the 3′ end of pre-miR-21. The pre-miRNA is intended to mimic the three-dimensional structure of the native pre-miR-21, such that the pre-miRNA of the invention is targeted into the exosomes during exosome biogenesis like the native pre-miR-21.

In some embodiments, the pre-miRNA comprising the structure A-B-C is defined as:

(A) the pre-miRNA 5′ end is substantially structurally and/or functionally similar to the 5′ end of pre-miR-21. The 5′ end of the pre-miR-21 contains a specific structure comprising wobble pair(s). The presence wobble pair(s) distorts the RNA deep groove and alters the secondary structure of the overall folded pre-miR-21. In the present invention, the 5′ end of the pre-miRNA 5′ end retains the secondary structure of the pre-miR-21 5′ end.

(B) the exogenous nucleotide sequence forms part of the stem structure of the stem-loop of the pre-miR-21 structural mimic. The exogenous nucleotide sequence can be from 19-25 nucleotide in length. The exogenous nucleotide sequence may retain the native structure of the stem of mature miR21-5p, with respect to length, wobble pairs, mismatches, deletions of the pre-miR-21-5p.

(C) the pre-miRNA 3′ end forms the loop structure at the 3′ end of the pre-miRNA. The 3′ end of the pre-miR-21 contains a specific structure comprising wobble pair(s). The presence wobble pair(s) distorts the RNA deep groove and alters the secondary structure of the overall folded pre-miR-21. The loop structure having an overall loop length of 4, 5, 6, 7, 8 or 9 nucleotides. Preferably 7 nucleotides in length. In the present invention, the 3′ end of the pre-miRNA 3′ end retains the secondary structure of the pre-miR-21 3′ end.

In a certain embodiment of the invention, the pre-miRNA comprising an exogenous nucleotide sequence comprises at least 50%, 55%, 60%, 65%, 70%, 75%, 80%, 85%, 90%, 95% or greater sequence identity to the sequence of native pre-miR-21-5p excluding the sequence for the mature miR-21-5p. The pre-miRNA is intended to mimic the overall three-dimensional structure of the native pre-miR-21, such that the pre-miRNA of the invention is targeted into the exosomes during exosome biogenesis like the native pre-miR-21.

By way of example, a % identity value may be determined by the number of matching identical nucleotides or amino acids divided by the sequence length for which the percent identity is being reported. Percentage (%) amino acid sequence similarity may be determined by the same calculation as used for determining % amino acid sequence identity, but may, for example, include conservative amino acid substitutions in addition to identical amino acids in the computation. Oligonucleotide alignment algorithms such as, for example, BLAST (GenBank; using default parameters) may be used to calculate sequence identity %. An alternative indication that two nucleotide sequences may be substantially identical is that the two sequences hybridize to each other under moderately stringent, or preferably stringent, conditions.

In an embodiment, the pre-miRNA of the invention (which may be referred to as a pre-miR-21 structural mimic), retains the biological function of the pre-miR-21. A “pre-miR-21 structural mimic” is a miRNA that is substantially structurally and/or functionally similar to pre-miR-21. The pre-miR-21 structural mimic may differ in sequence but retains one or more biological function of pre-miR-21. By “retains one or more biological function” it is meant that the mimic retains at least 95%, 90%, 80% 70%, 60%, 50% of the biological function in question. In a particular embodiment, the biological function in question may be sorting of the pre-miR-21 structural mimic to exosomes. In this embodiment, the pre-miRNA of the invention is an miRNA that may differ in sequence but shares structurally and/or functionally similar, primary, secondary and/or tertiary structure as the native miR21-5p such that it is packaged into exosomes at least 95%, 90%, 80% 70%, 60%, 50% of the level of native pre-miR-21.

In another embodiment, the pre-miRNA of the invention shares structurally and/or functionally similar, primary, secondary and/or tertiary structure such that it is able to bind to RBPs with a binding affinity of at least 95%, 90%, 80% 70%, 60%, 50% of the binding affinity of native pre-miR-21. In another embodiment, the pre-miRNA of the invention shares structurally and/or functionally similar, primary, secondary and/or tertiary structure such that it is able to bind to miRNA processing enzymes with a binding affinity of at least 95%, 90%, 80% 70%, 60%, 50% of the binding affinity of native pre-miR-21. Examples of miRNA processing enzymes include Ran-GTP, Exportin-5, and Dicer. Binding affinity of the pre-miRNA can be measured by, for example, Fluorescence Polarisation (FP), Fluorescence Resonance Energy Transfer (FRET), and Surface Plasmon Resonance (SPR).

The amount of exogenous nucleotide sequence present in a purified exosomes can be quantified using qPCR or other quantitative method such as ddPRC. The structure of pre-miRNA can be determined by methods such as X-ray crystallography, nuclear magnetic resonance (NMR) spectroscopy, cryo-electron microscopy (cyro-EM), chemical/enzymatic probing, thermal denaturation, and mass spectrometry.

### MIR-21 Expression Cassette

The invention provides an expression cassette comprising a pre-miRNA comprising an exogenous nucleotide sequence that is based on the structure of pre-miR-21, termed a pre-miR-21 structural mimic.

In some embodiments, the invention provides an expression cassette comprising one or more of, or all of a promoter region that can be a naturally-occurring sequence or a designed regulatory sequence, a 5′ sequence upstream, a 3′ sequence downstream to the miRNA containing transcription starting points, stabilizing sequence(s), enhancer(s), and/or repressor sequence(s) to control the transcription of the miRNA; and a pre-miRNA comprising an exogenous nucleotide sequence that is based on the structure of pre-miR-21, termed a pre-miR-21 structural mimic. In an aspect of the invention, a cassette comprising the pre-miRNA of the invention is provided. In a particular embodiment, the cassette comprises: 5′ pri_5miR21 sequence; a pre-miR-21 structural mimic; and a 3′ pri_miR21 sequence (in that order). In certain embodiments, the miRNA expression cassette preserves the original sequences and length of the pri- and pre-mi-21 including the loop, miss-pairs, Wobble pairs and lack of base-pairing of within the original naturally occurring miRNA. This is intended to maintain the structure of pre-miR-21 to facilitate shuttling of the exogenous nucleotide sequence into exosomes.

Typically this cassette can be introduced via a cloned DNA sequence within a DNA vector transfected into the producer line of choice with the purpose of generating a genetically modified cell line that transcribes the RNA moieties described herein using the cells internal transcription machinery. The cassette or a part of it can also be introduced in the endogenous locus of the gene MIR21 by using any technology allowing genetic manipulation of this locus, such as CRISPR, in order to replace a part of it for the expression of a nucleic acid of interest. A “vector” is a small piece of DNA, taken from a virus, a plasmid, or the cell of a higher organism, that can be stably maintained in an organism, and into which a foreign DNA fragment can be inserted for cloning purposes. The design of such an expression vector is outlined in Example 3.

The pre-miRNA may be introduced to the locus for MIR21 to replace the natural occurring miR-21 sequence by using any gene editing technology such as CRISPR/Cas, TALENs or zinc fingers or prime editing so allowing specific gene recombination. The pre-miRNA may also be introduced to the locus for MIR21 to replace the natural occurring miR-21 sequence by using any technology allowing specific gene recombination.

Alternatively, the pre-miRNA can be transfected directly in the form of the stem-loop structure or generated ex vivo. This can be achieved by either using RNA constructs generated in vitro that use a RNA-polymerase promoter and a DNA guide strand with a transcription mix using any acceptable in vitro methodology, or by chemically synthesising the desired construct using any number of known oligosynthesis methodologies. By synthetically producing the pre-miRNAs it would also be possible to generate chemically modified forms of the natural nucleotides and/or non-natural nucleotides in the constructs. This would allow for the use of modified siRNA, miRNA and other RNA constructs such as anti-miRs that have improved systemic stability and/or decreased immunogenicity and/or more selective target binding with alterations in sequence at the seed sequence. Such ex vivo RNA constructs would then be introduced to the producer cell line via any number of transfection methods available (e.g. lipofectamine, electroporation, or any other means of transfection or nucleic acid delivery).

By using the pre-miRNA expression cassette of the invention, the inventors have generated a functional pre-miRNA comprising an exogenous nucleotides sequence in situ that has been shown to be transported into exosomes at during their biogenesis within the cell. Moreover, it has been observed that this exosome-encapsulated exogenous nucleotide sequence of choice is also functional in a recipient cell line and able to be delivered to a recipient cell to modulate target gene activity, for example, down-regulation in a target cell.

Accordingly, using this invention:
i) an exogenous nucleotide sequence (for example a target gene silencing or gene editing/replacement oligomeric single stranded RNA sequence) can be packaged within an exosomal loading cassette;
ii) such a cassette complete with the exogenous nucleotide sequence can be embodied within a mammalian expression vector or synthesised as a complete stem loop structure ex vivo;
iii) either expression vector or stem loop as described above can be transfected in to any chosen target cell (e.g. using electroporation or lipoefectamine, or via another exosome), the choice of such cell can be dictated by the desired tissues that the user requires the final exosomal delivery vehicle to target and/or if the therapeutic target is to cross the blood brain barrier (in which case a neural producer cell may be chosen);
iv) the exogenous nucleotide sequences can be targeted for processing in to exosomes by using the producer cells natural exosome shuttling mechanisms, irrespective of whether the cells are expressing Argonaut proteins or not, and irrespective of whether DICER is functional or not within such cells;
v) exosomes loaded with the desired exogenous nucleotide sequence can be harvested from the conditioned media from producer cells using any number of known exosome harvesting methods including but not limited to ultracentrifugation, PEG precipitation, TFF, affinity chromatography etc.;
vi) exosomes can deliver packaged exogenous nucleotide sequence into human cells in vitro and in vivo, and that in certain cases depending on the originating producer cell, the exosomes can be target to particular tissues when delivered systemically; and
vii) contacting the target cells with the exosomes delivers the exogenous nucleotide sequence to the target cells and modulates gene activity.

The invention enables the loading of any gene-silencing oligonucleotide including siRNA, miRNA, or antisense oligonucleotide, and any gene editing tool such as a CRISPR RNA guide strand, and any other single stranded RNA molecule. These nucleotide sequences are intended to be loaded in to an exosome of choice and produced at scale using cell culturing and exosome purification techniques.

The method of the invention discloses loading of exosomes with exogenous nucleotide sequences by engineering the producer cell via expression vectors. The method is also applicable to modification through introduction of native or synthetic constructs comprising the actual miR-21 pri-microRNA plus RNA cargo into the producer cell directly. The constructs can be transfected into producer cells by any means.

The invention has an advantage in that it enables the production of large quantities of exogenous nucleotide sequence loaded into exosomes. A particular advantage of the invention is that the method of production of such exogenous nucleotide sequence, e.g. therapeutic-miRNA, loaded exosomes is reduced to the harvest of conditioned media, purification and validation. Therefore, reducing the time and costs associated with other methods for loading exogenous nucleotides sequences such as miRNA into exosomes.

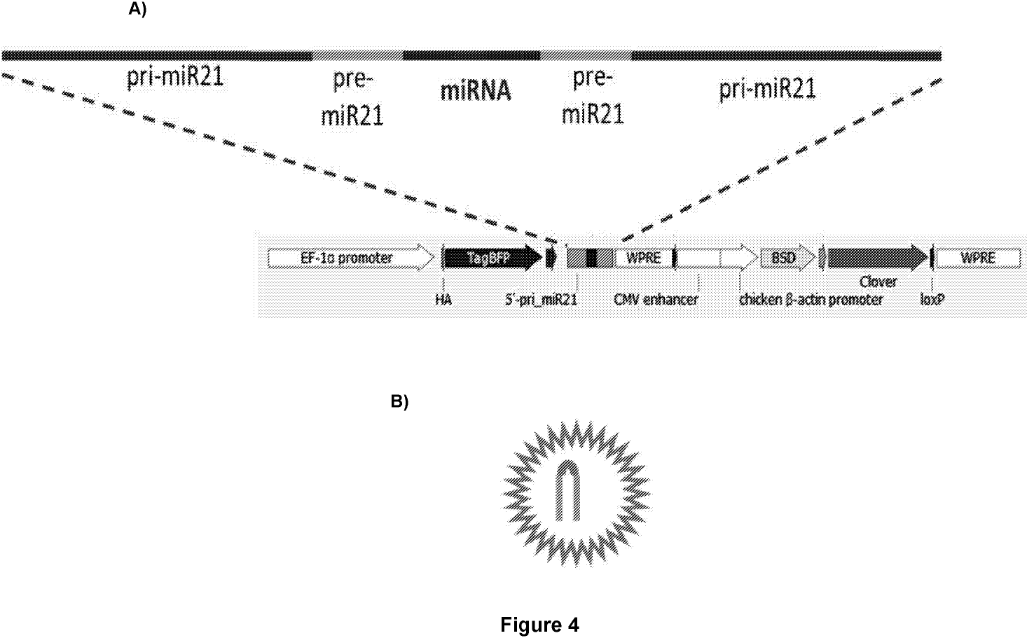

The pre-miRNA expression cassette and the methods disclosed herein can be used to deliver any exogenous or endogenous single stranded nucleotide sequence for gene silencing or gene editing/replacement. Therefore, providing the means for generating a therapeutic moiety encapsulated or associated with an exosome for in vivo delivery (FIG. 4). The benefits of this system include improved tissue targeting, lower immunogenicity, and protection from RNAse and other factors that may otherwise accelerate degradation of the ‘naked’ therapeutic moiety in vivo. The harvested loaded exosomes can also be used to deliver exogenous nucleotides sequences to cells in vitro in order to engineer cells that can be used in transplantation techniques such as CAR-T cell therapies.

### Exogenous Nucleotide Sequences

An “exogenous nucleotide sequence” is nucleotide sequence that is not naturally found in native pre-miR-21-5p. In some embodiments, the exogenous nucleotide sequence modulates gene activity in a target cell.

In an embodiment of the invention, the exogenous nucleotide sequence is a sequence for an shRNA, siRNA, miRNA, anti-miR, ASO, CRISPR guide RNA, or other exogenous nucleotide sequence. In a further embodiment of the invention, the exogenous nucleotide sequence is an miRNA e.g. miR-146-b or miR-1246, or an siRNA.

In certain embodiments, the exogenous nucleotide sequence may be therapeutic. Therapy with nucleotide sequences can comprise DNA or RNA, or an RNA-DNA hybrid.. DNA therapeutics comprise antisense oligonucleotides, DNA aptamers, and gene therapy. RNA therapeutics comprise RNAi, and guide RNA for CRISPR. In some embodiments, the nucleotide sequence of the invention is a therapeutic RNA.

Anti-sense oligonucleotides (ASOs) are single-stranded sequences of 8-50 base pairs in length, binding to the target mRNA by means of standard Watson-Crick base pairing. After an ASO binds with the mRNA, either the target complex will be degraded by endogenous cellular RNase H or a functional blockade of mRNA occurs due to steric hindrance. DNA or RNA aptamers, also called ‘chemical antibodies’, are single-stranded synthetic DNA or RNA molecules, 56-120 nucleotides long, that can bind the nucleotide coding for proteins with high affinity and thus serve as decoys. DNA aptamers are short single-stranded oligonucleotide sequences similar to ASO with very high affinity for the target nucleic acids through structural recognition (Sridharan and Gogtay. 2016).

RNA interference (RNAi) is a regulatory mechanism in eukaryotic cells that use small double-stranded RNA (dsRNA) molecules to regulate gene expression. RNAi is a mechanism whereby approximately 21 nucleotide long double-stranded RNA molecules can potently silence or repress expression of specific genes having complementary mRNA sequence. Two types of small RNA molecules are central to RNAi. These are micro RNA (miRNA) and small interfering RNA (siRNA). In humans, genes expression is reduced by cleaving and degrading RNA perfectly complementary to the gene silencing nucleic acid (i.e. siRNA guide strand), or repressing the translation of imperfectly complementary mRNA (such as in the case of miRNA gene silencing nucleic acids). In humans, the primary class of small RNA genes silencers are called microRNAs (miRNAs), which regulate large gene networks by repressing translation of mRNA with partially complementary binding sites.

The CRISPR-Cas9 system allows for targeted editing of DNA. The system is targeted to the DNA via association with a guide RNA (gRNA) molecule, which binds to the targeted DNA through base complementarity and enables precise DNA cleavage in both strands. The end result of Cas9-mediated DNA cleavage is a double-strand break (DSB) within the target DNA that is repaired by the intracellular repairing mechanism of the cell by the efficient but error-prone non-homologous end joining (NHEJ) pathway, or by the less efficient but high-fidelity homology directed repair (HDR) pathway. Replacement genetic sequences can also be cotransfected eg using AAVs to encode WT replacement sequences when carrying out augmented gene therapy for autosomally dominant inherited genetic diseases. Both repairing mechanisms can be exploited for different outcomes. The success of the CRISPR-Cas9 system therefore hinges on the correct identification of the optimal target-site and subsequent design of the complimentary gRNA (Wilson, et al. 2018).

Delivery of RNA interference-based therapeutics has presented a significant challenge. Many strategies to deliver RNAi therapeutics have been tested, including lipid particles, siRNA-modification, nanoparticles, and aptamers (Whitehead, et al. 2009; Kanasty, et al. 2013). However, in many cases delivery has been unsuccessful, and there remains a roadblock to the delivery of RNAi-based therapeutics (Kanasty, et al. 2013; Tatiparti, et al. 2017). Exosomes offer a solution to this roadblock in the delivery of RNAi-based therapeutics. However, a major roadblock to using exosomes or exosome-like vesicles as drug delivery vehicles for therapeutic nucleotide sequences such as gene silencing nucleotide sequence is the ability to package siRNA/RNAi/miRNA, or other nucleotide sequence of interest, into exosomes. Exosomes have a highly selective content of both proteins and RNA as compared to the cells that produce them. The present invention overcomes this problem by providing an improved method of loading exosomes with a pre-miRNA comprising an exogenous nucleotide sequence or the gene edition of the locus MIR21 in order to express the pre-miRNA.

### Exosomes

The invention provides an exosome loaded with a nucleic acid construct having the structure and/or function of pre-miR-21 and comprising an exogenous nucleotide sequence.

Exosomes originate in the endosomal compartment and are secreted when the Multivesicular Bodies (MVBs) fuse with the plasma membrane.

Exosomes are a type of microparticle. A “microparticle” is an extracellular vesicle of 30 to 1000 nm diameter that is released from a cell. It is limited by a lipid bilayer that encloses biological molecules. The term “microparticle” is known in the art and encompasses a number of different species of microparticle, including a membrane particle, membrane vesicle, microvesicle, exosome-like vesicle, exosome, ectosome-like vesicle, ectosome or exovesicle. The different types of microparticle are distinguished based on diameter, subcellular origin, their density in sucrose, shape, sedimentation rate, lipid composition, protein markers and mode of secretion (i.e. following a signal (inducible) or spontaneously (constitutive)). Four of the common microparticles and their distinguishing features are described in Table 1, below.

TABLE 1

Various Microparticles

| | | | | | |
|---|---|---|---|---|---|
| Microparticle | Size | Shape | Markers | Lipids | Origin |
| Microvesicles | 100-1000 nm | Irregular | Integrins, selectins, CD40 ligand | Phosphatidylserine | Plasma membrane |
| Exosome-like vesicles | 20-50 nm | Irregular | TNFRI | No lipid rafts | MVB from other organelles |
| Exosomes | 30-100 nm; (<200 nm) | Cup shaped | Tetraspanins (e.g. CD63, CD9), Alix, TSG101, ESCRT | Cholesterol, sphingomyelin, ceramide, lipid rafts, phosphatidylserine | Multivesicular endosomes |
| Membrane particles | 50-80 nm | Round | CD133, no CD63 | Unknown | Plasma membrane |

Exosomes are typically defined as having a diameter of 30-100 nm, but more recent studies confirm that exosomes can also have a diameter between 100 nm and 200 nm, (e.g. Katsuda, et al. Proteomics 2013, and Katsuda, et al. Scientific Reports 2013). Accordingly, exosomes typically have a diameter between 30 nm and 150 nm. The diameter can be determined by any suitable technique, for example electron microscopy or dynamic light scattering.

Exosomes are thought to play a role in intercellular communication by acting as vehicles between a donor and recipient cell through direct and indirect mechanisms. Direct mechanisms include the uptake of the exosome and its donor cell-derived components (such as proteins, lipids or nucleic acids) by the recipient cell, the components having a biological activity in the recipient cell. Indirect mechanisms include exosome-recipient cell surface interaction, and causing modulation of intracellular signalling of the recipient cell. Hence, exosomes may mediate the acquisition of one or more donor cell-derived properties by the recipient cell.

In some embodiments of the invention, the exosomes loaded with a pre-miRNA comprising an exogenous nucleotide sequence are isolated. The term “isolated” indicates that the exosome or exosome population to which it refers is not within its natural environment. The exosome or exosome population has been substantially separated from surrounding tissue. In some embodiments, the exosome or exosome population is substantially separated from surrounding tissue if the sample contains at least about 75%, in some embodiments at least about 85%, in some embodiments at least about 90%, and in some embodiments at least about 95% exosomes. In other words, the sample is substantially separated from the surrounding tissue if the sample contains less than about 25%, in some embodiments less than about 15%, and in some embodiments less than about 5% of materials other than the exosomes. Such percentage values refer to percentage by weight. The term encompasses exosomes that have been removed from the organism from which they originated, and exist in culture. The term also encompasses exosomes that have been removed from the organism from which they originated, and subsequently re-inserted into an organism.

Exosomes can be secreted from virtually any cell type. In some embodiments to exosomes are secreted from stem cells, hIPSCs, tissue stem cells, differentiated cells from any of those or dendritic cells. In a certain embodiment, the exosomes are secreted from stem cells. Stem cells naturally produce exosomes by the fusion of intracellular multivesicular bodies (which contain microparticles) with the cell membrane and the release of the exosomes into the extracellular compartment.

In another embodiment, the stem cell is a neural stem cell. Neural stem cells (NSCs) are self-renewing, multipotent stem cells that generate neurons, astrocytes and oligodendrocytes (Kornblum, 2007). In some embodiments, the neural stem cell line may be the “CTX0E03” cell line, the “STR0C05” cell line, the “HPC0A07” cell line or the neural stem cell line disclosed in Miljan et al. 2009. The neural stem cell may be any of the neural stem cells described herein, for example the CTX0E03 conditionally-immortalised cell line, which is clonal, standardised, shows clear safety in vitro and in vivo and can be manufactured to scale thereby providing a unique resource for stable exosome production. Alternatively, the neural stem cells may be neural retinal stem cell lines, optionally as described in US 7514259 (which is incorporated by reference).

A neural stem cell exosome is an exosome that is produced by a neural stem cell. Typically, the exosome is secreted by the neural stem cell. Exosomes from other cells, such as mesenchymal stem cells, are known in the art.

The neural stem cell that produces the exogenous nucleotide sequence loaded exosomes of the invention can be a fetal, an embryonic, or an adult neural stem cell, such as has been described in US5851832, US6777233, US6468794, US5753506 and WO-A-2005121318 (incorporated by reference). The fetal tissue may be human fetal cortex tissue. The cells can be selected as neural stem cells from the differentiation of induced pluripotent stem (iPS) cells, as has been described by Yuan, et al. (2011) or a directly induced neural stem cell produced from somatic cells such as fibroblasts (for example by constitutively inducing Sox2, Klf4, and c-Myc while strictly limiting Oct4 activity to the initial phase of reprogramming as recently by Their, et al. 2012). Human embryonic stem cells may be obtained by methods that preserve the viability of the donor embryo, as is known in the art (e.g. Klimanskaya et al. 2006, and Chung, et al. 2008). Such non-destructive methods of obtaining human embryonic stem cell may be used to provide embryonic stem cells from which microparticles of the invention can be obtained. Alternatively, the exogenous nucleotide sequence loaded exosomes of the invention can be obtained from adult stem cells, iPS cells or directly-induced neural stem cells. Accordingly, the exogenous nucleotide sequence loaded exosomes of the invention can be produced by multiple methods that do not require the destruction of a human embryo or the use of a human embryo as a base material.

Typically, the neural stem cell population from which the exosomes are produced is substantially pure. The term “substantially pure” as used herein, refers to a population of stem cells that is at least about 75%, in some embodiments at least about 85%, in some embodiments at least about 90%, and in some embodiments at least about 95% pure, with respect to other cells that make up a total cell population. For example, with respect to neural stem cell populations, this term means that there are at least about 75%, in some embodiments at least about 85%, in some embodiments at least about 90%, and in some embodiments at least about 95% pure, neural stem cells compared to other cells that make up a total cell population. In other words, the term “substantially pure” refers to a population of stem cells of the present invention that contain fewer than about 25%, in some embodiments fewer than about 15%, and in some embodiments fewer than about 5%, of lineage committed cells in the original unamplified and isolated population prior to subsequent culturing and amplification.

An exosome comprises at least one lipid bilayer which typically encloses a milieu comprising lipids, proteins and nucleic acids. The nucleic acids may be deoxyribonucleic acid (DNA) and/or ribonucleic acid (RNA). RNA may be messenger RNA (mRNA), micro RNA (miRNA) or any miRNA precursors, such as pri-miRNA, pre-miRNA, and/or small nuclear RNA (snRNA).

A stem cell-derived exosome retains at least one biological function of the stem cell from which it is derived. Biological functions that may be retained include the ability to promote angiogenesis and/or neurogenesis, the ability to effect cognitive improvement in the brain of a patient that has suffered a stroke, or the ability to accelerate blood flow recovery in peripheral arterial disease. For example, CTX0E03 cells are known to inhibit T cell activation in a PBMC assay and, in one embodiment, the microparticles of the invention retain this ability to inhibit T cell activation in a PBMC assay. PBMC assays are well-known to the skilled person and kits for performing the assay are commercially available.

Some exosomes of the invention express the CD133 surface marker. Other exosomes of the invention do not express the CD133 surface marker. “Marker” refers to a biological molecule whose presence, concentration, activity, or phosphorylation state may be detected and used to identify the phenotype of a cell.

Exosomes are endosome-derived lipid microparticles of typically 30-100 nm diameter and sometimes between 100 nm and 200 nm diameter that are released from the cell by exocytosis. Exosome release occurs constitutively or upon induction, in a regulated and functionally relevant manner. During their biogenesis, exosomes incorporate a wide range of cytosolic proteins (including chaperone proteins, integrins, cytoskeletal proteins and the tetraspanins) and genetic material. Consequently, exosomes are considered to be inter-cellular communication devices for the transfer of proteins, lipids and genetic material between cells, in the parent cell microenvironment and over considerable distance. Although the invention is not bound by this theory, it is possible that the exosomes are responsible for the efficacy of the neural stem cells. Therefore, exosomes from neural stem cells are themselves expected to be therapeutically efficacious.

In one embodiment, isolated or purified exogenous nucleotide sequence loaded exosomes are also loaded with one or more exogenous nucleic acids, lipids, proteins, drugs or prodrugs which are intended to perform a desired function in a target cell. This does not require manipulation of the stem cell and the exogenous material can optionally be directly added to the exosomes. For example, exogenous peptides or proteins can be introduced into the exosomes by electroporation. The microparticles can then be used as vehicles or carriers for the exogenous material. In this way, microparticles can be used as vehicles to deliver one or more agents, typically therapeutic or diagnostic agents, to a target cell.

### Neural Stem Cells

The neural stem cell that produces the exosome may be a stem cell line, i.e. a culture of stably dividing stem cells. A stem cell line can to be grown in large quantities using a single, defined source. Immortalisation may arise from a spontaneous event or may be achieved by introducing exogenous genetic information into the stem cell which encodes immortalisation factors, resulting in unlimited cell growth of the stem cell under suitable culture conditions. Such exogenous genetic factors may include the gene “myc”, which encodes the transcription factor Myc. The exogenous genetic information may be introduced into the stem cell through a variety of suitable means, such as transfection or transduction. For transduction, a genetically engineered viral vehicle may be used, such as one derived from retroviruses, for example lentivirus.

Additional advantages can be gained by using a conditionally immortalised stem cell line, in which the expression of the immortalisation factor can be regulated without adversely affecting the production of therapeutically effective microparticles. This may be achieved by introducing an immortalisation factor which is inactive unless the cell is supplied with an activating agent. Such an immortalisation factor may be a gene such as c-mycER. The c-MycER gene product is a fusion protein comprising a c-Myc variant fused to the ligand-binding domain of a mutant estrogen receptor. c-MycER only drives cell proliferation in the presence of the synthetic steroid 4-hydroxytamoxifen (4-OHT) (Littlewood, et al.1995). This approach allows for controlled expansion of neural stem cells in vitro, while avoiding undesired in vivo effects on host cell proliferation (e.g. tumour formation) due to the presence of c-Myc or the gene encoding it in microparticles derived from the neural stem cell line. A suitable c-mycER conditionally immortalized neural stem cell is described in U.S. Pat. 7416888. The use of a conditionally immortalised neural stem cell line therefore provides an improvement over existing stem cell microparticle isolation and production.

Preferred conditionally-immortalised cell lines include the CTX0E03, STR0C05 and HPC0A07 neural stem cell lines, which have been deposited by the applicant at the European Collection of Animal Cultures (ECACC), Vaccine Research and Production laboratories, Public Health Laboratory Services, Porton Down, Salisbury, Wiltshire, SP4 0JG, with Accession No. 04091601 (CTX0E03); Accession No.04110301 (STR0C05); and Accession No.04092302 (HPC0A07). The derivation and provenance of these cells is described in EP1645626 B1. The advantages of these cells are retained by exosomes produced by these cells.

The cells of the CTX0E03 cell line may be cultured in the following culture conditions:
Human Serum Albumin 0.03%
Human Transferrin 5 µg/ml
Putrescine Dihydrochloride 16.2 µg/ml
Human recombinant Insulin 5 µ/ml
Progesterone 60 ng/ml
L-Glutamine 2 mM
Sodium Selenite (selenium) 40 ng/ml

Plus basic Fibroblast Growth Factor (10 ng/ml), epidermal growth factor (20 ng/ml) and 4-hydroxytamoxifen 100 nM for cell expansion. The cells can be differentiated by removal of the 4-hydroxytamoxifen. Typically, the cells can either be cultured at 5% CO₂/37° C. or under hypoxic conditions of 5%, 4%, 3%, 2% or 1% O₂. These cell lines do not require serum to be cultured successfully. Serum is required for the successful culture of many cell lines, but contains many contaminants including its own exosomes. A further advantage of the CTX0E03, STR0C05 or HPC0A07 neural stem cell lines, or any other cell line that does not require serum, is that the contamination by serum is avoided.

The cells of the CTX0E03 cell line (and microparticles derived from these cells) are multipotent cells originally derived from 12 week human fetal cortex. The isolation, manufacture and protocols for the CTX0E03 cell line is described in detail by Sinden, et al. (U.S. Pat. 7,416,888 and EP1645626 B1). The CTX0E03 cells are not “embryonic stem cells”, i.e. they are not pluripotent cells derived from the inner cell mass of a blastocyst; isolation of the original cells did not result in the destruction of an embryo.

CTX0E03 is a clonal cell line that contains a single copy of the c-mycER transgene that was delivered by retroviral infection and is conditionally regulated by 4-OHT (4-hydroxytamoxifen). The c-mycER transgene expresses a fusion protein that stimulates cell proliferation in the presence of 4-OHT and therefore allows controlled expansion when cultured in the presence of 4-OHT. This cell line is clonal, expands rapidly in culture (doubling time 50-60 hours) and has a normal human karyotype (46 XY). It is genetically stable and can be grown in large numbers. The cells are safe and non-tumorigenic. In the absence of growth factors and 4-OHT, the cells undergo growth arrest and differentiate into neurons and astrocytes.

The development of the CTX0E03 cell line has allowed the scale-up of a consistent product for clinical use. Production of cells from banked materials allows for the generation of cells in quantities for commercial application (Hodges, et al. 2007).

The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin, collagen matrices and/or other proteins forming any extracellular matrix. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for bacterial culture is a medium. Similarly, a powder mixture that when mixed with water or other liquid becomes suitable for cell culture may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the invention while maintaining their self-renewal capability. Examples of basal media include, but are not limited to, Eagles Basal Medium, Minimum Essential Medium, Dulbecco’s Modified Eagle’s Medium, Medium 199, Nutrient Mixtures Ham’s F-10 and Ham’s F-12, McCoy’s 5A, Dulbecco’s MEM/F-I 2, RPMI 1640, and Iscove’s Modified Dulbecco’s Medium (IMDM).

### Transfection of Cells to Produce Exosomes

Transfection of the exosome-producer cells with the nucleic acid construct of the invention can be carried out using multiple methods. Such methods include, but are not limited to, cationic lipid transfection, electroporation, viral transfection, and calcium phosphate transfection.

The nucleic acid construct of the invention comprises the pre-miRNA of the invention, or the cassette of the invention, or the vector of the invention.

In some embodiments, cationic lipid transfection is used to transfect the nucleic acid construct into the exosome producer cell. In a further embodiment, cationic lipid transfection is carried out using reagents such as, DOTMA (N-[1-(2,3,-dioleyloxy)propyl]-N,N,N-trimethylammonium chloride), TRANSIT®, X-TREMEGENE™ transfection reagent LIPOFECTIN®, LIPOFECTAMINE®, and OLIGOFECTAMINE®.

In some embodiments, electroporation is used to transfect the nucleic acid construct into the exosome producer cell. Electroporation involves exposure of cell membranes to high-intensity electric pulses which cause temporary destabilisation making the cell highly permeable, allowing the entry of exogenous molecules. Electroporation is an easy, non-chemical technique that can yield high transformation efficiencies in various cell types.

In some embodiments, viral transfection is used to transfect the nucleic acid construct into the exosome producer cell. This method involves the use of viral vectors to deliver nucleic acids into cells. Viral delivery systems such as lentiviral, adenoviral, adeno-associated viral systems and oncoretroviral vectors can be used for transferring nucleotide sequences, even in hard-to-transfect cells.

In some embodiments, calcium phosphate is used to transfect the nucleic acid construct into the exosome producer cell. The calcium phosphate transfection technique involves the precipitation of DNA and calcium phosphate. The precipitation is facilitated by mixing a HEPES-buffered saline solution, having sodium phosphate, with calcium chloride solution and DNA2.

The amount of transfected nucleic acid construct present in a purified exosomes can be quantified using qPCR (Example 1) or other techniques that allow RNA quantification such as FISH/Scope, droplet-digital PCR or RNAseq.

### Exosome Purification

Exosomes of the invention may be purified using known exosome purification techniques. For example, exosomes can be purified by Tangential Flow Filtration (TFF) or ultracentrifugation, e.g. 100000 x g for 1-2 hours. Alternative or additional methods for purification of may be used, such as antibody-based methods, e.g. immunoprecipitation, magnetic bead purification, resin-based purification, using specific antibodies.

The exosomes can be subsequently quantified and characterised as described in WO-A-2013/150303 and WO-A-2014/013258 (incorporated by reference).

### Packaging of Exogenous Nucleotide Sequences Within Exosomes for Delivery to Target Cells

Exosomes represent a particularly interesting delivery option for the delivery of exogenous nucleotide sequences to target cells. The present invention capitalises on an endogenous system for intercellular communication thereby providing a system for the delivery exogenous nucleotide sequence into target cells. Exosomes can be modified to target a variety of specific cell types and tissues. Exosomes secreted from different cell types express different proteins on their surface that may target them to different target cells.

The invention therefore provides an exosome loaded an exogenous nucleic acid cargo, for example an exosome loaded with a pre-miRNA comprising an exogenous nucleotide sequence. The invention also provides a method of delivering an exogenous nucleotide sequence to a target cell, using an exosome loaded e.g. with the pre-miRNA, wherein the method comprises contacting the target cell with the loaded exosomes. In some embodiments, a targeting moiety is expressed or conjugated to the surface of the exosomes to target the exosome comprising an exogenous nucleotide sequence to a particular cell type.

A target cell is a cell in which the exogenous nucleotide sequence is intended to modulate gene activity. A target cell may be a cell in vitro or in vivo. In some embodiments the target cell is a cancer cell, a stem cell, an immune cell. In other embodiments, the target cell is a neuronal cell, a stromal cell, or a muscle cell.

### Assessing Gene Modulation in the Target Cell

There are many methods that can be used to assess modulation of gene activity in a target cell. The following methods are to serve as examples of methods and are not limiting. In some embodiments, the pre-miRNA of the invention results in a reduction of expression a target gene in a target cell.

Western blot is a widely used analytical technique used in molecular biology to detect specific protein molecules from a mixture of proteins. Western blotting can be used to measure the amount of protein expression. The method includes, preparing the protein sample by mixing it with a detergent, such as SDS, separation of the proteins using gel electrophoresis, transferring the proteins from the gel to a blotting membrane, blocking the membrane, incubating with a primary antibody, incubating with secondary antibody linked to a reporter enzyme that produces colour or light, and detecting this colour or light. In some embodiments quantitative western blotting can be carried out to assess modulation of gene activity. In some embodiments, a reduction in protein quantity is indicative of down-regulation of a gene. In another embodiment, an increase in protein quantity is indicative of up-regulation of a gene.

Changes in gene expression in cells can be measured using quantitative polymerase chain reaction (PCR). In some embodiments, quantitative PCR (qPCR) can be carried out to assess modulation of gene activity. Such a method may include isolating total RNA from the target cell, performing cDNA synthesis, running qPCR reactions, and analysing the results from qPCR using relative quantification (Fleige and Pfaffl. 2006). In some embodiments, the qPCR is carried out using SYBR-green or TaqMan/TaqPath probes. In some embodiments, a reduction in RNA is indicative of down-regulation of a gene. In another embodiment, an increase in RNA is indicative of up-regulation of a gene.

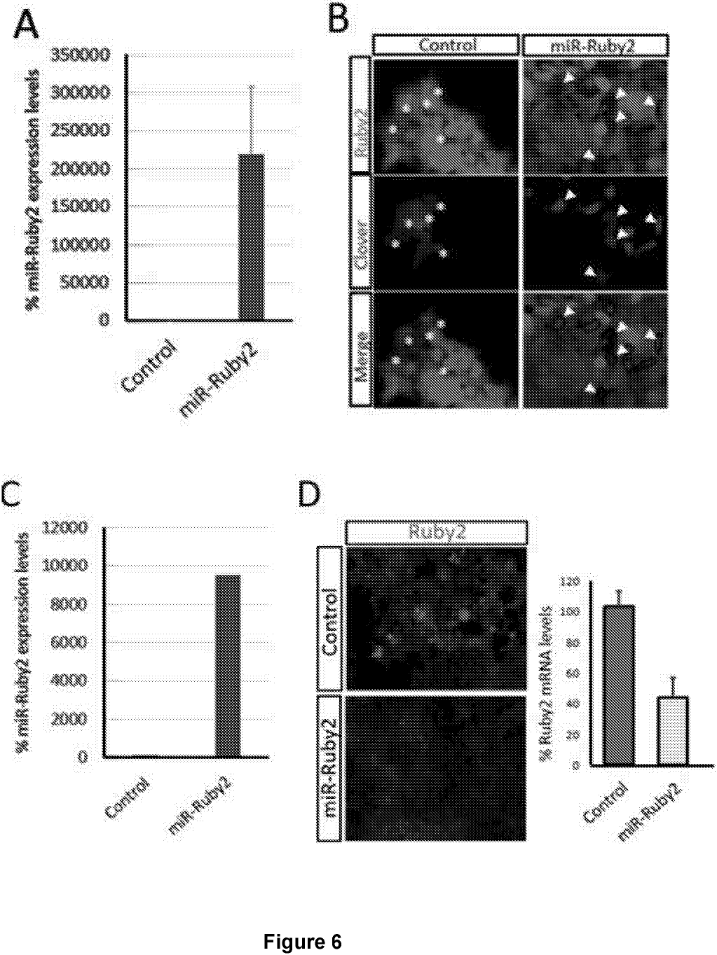

In some embodiments, reporter assays are used to assess gene modulation. In a further embodiment, the reporter assay is a fluorescence reporter assay using a fluorescent protein such as tagBFP, GFP, eGFP, YFP, mcherry, Ruby2, mOrange, Citrine, Clover, and mTurquoise. A fluorescent protein reporter system is described in Example 1, and FIGS. 5 and 6. Changes in expression of the fluorescence protein or fusion protein can be measured using flow cytometry, microscopy, or high throughput quantitative microscopy. In some embodiments, a reduction in fluorescent signal is indicative of down-regulation of a gene. In another embodiment, an increase in fluorescent signal is indicative of up-regulation of a gene.

In another embodiment, the reporter assay uses a luciferase-based system. In some embodiments, the luciferase reporter that can be used is Cypridina Luciferase, Gaussia Luciferase, Gaussia-Dura Luciferase, Green Renilla Luciferase, Red Firefly Luciferase, Renilla Luciferase, Nano-Luc Luciferase or TurboLuc Luciferase. In the luciferase-based system a change in gene expression is measured using a luminometer or modified optical microscopes (McClure, et al. 2011), but can also been measured by other means such as qPCR, Western Blot, immunochemistry or flow cytometry. In some embodiments, a reduction in signal is indicative of down-regulation of a gene. In another embodiment, an increase in signal is indicative of up-regulation of a gene.

In some embodiments of the invention, the nucleic acid construct of the invention results in a reduction of gene expression by at least 10%, 20%, 30%, 40%, 50%, 60%, 70%, 80%, 90% or greater. Typically, gene expression is reduced by 60% to 100%. In some embodiments, of the invention, the nucleic acid construct of the invention results in an increase in gene expression by at least 10%, 20%, 30%, 40%, 50%, 60%, 70%, 80%, 90% or greater. Typically, gene expression is increased by 60% to 100%.

### Therapeutic Uses

The exosomes loaded with pre-miRNA comprising an exogenous nucleotide sequence may be useful in the treatment or prophylaxis of disease. Accordingly, the invention includes a method of treating or preventing a disease or disorder in a patient using an exosome loaded with pre-miRNA comprising an exogenous nucleotide sequence. The term “patient” includes human and other mammalian subjects that receive either prophylactic or therapeutic treatment.

In some embodiments, the exogenous nucleic acid is a therapeutic RNA sequence for an RNA therapeutic. Examples of RNA therapeutics that are currently in clinical trials include RNA therapeutics for cancer, liver fibrosis, glaucoma, cystic fibrosis, ulcerative colitis, Hepatitis B infection, Type 2 diabetes, Duchenne muscular dystrophy, asthma, and HIV infections (Kaczmarek, et al. 2017). Such RNA therapeutics may benefit from the invention, by providing a delivery system to target cells by packaging such therapeutic RNAs into exosomes.

The invention also provides a method for treating or preventing a disease or condition comprising administering an effective amount of the exosome of the invention, thereby treating or preventing the disease. The exosomes of the invention can be used to treat the same diseases as the stem cells from which they are obtained.

In prophylactic applications, pharmaceutical compositions or medicaments are administered to a patient susceptible to, or otherwise at risk of, a particular disease in an amount sufficient to eliminate or reduce the risk or delay the outset of the disease. In therapeutic applications, compositions or medicaments are administered to a patient suspected of, or already suffering from such a disease in an amount sufficient to cure, or at least partially arrest, the symptoms of the disease and its complications. An amount adequate to accomplish this is defined as a therapeutically-or pharmaceutically-effective dose. In both prophylactic and therapeutic regimes, agents are typically administered in several dosages until a sufficient response has been achieved. Typically, the response is monitored and repeated dosages are given if the response starts to fade.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic.

As used herein, the terms “treat”, “treatment”, “treating” and “therapy” when used directly in reference to a patient or subject shall be taken to mean the amelioration of one or more symptoms associated with a disorder, or the prevention or prophylaxis of a disorder or one or more symptoms associated with a disorder. The disorders to be treated include, but are not limited to, a degenerative disorder, a disorder involving tissue destruction, a neoplastic disorder, an inflammatory disorder, an autoimmune disease or an immunologically mediated disease including rejection of transplanted organs and tissues. Amelioration or prevention of symptoms results from the administration of the microparticles of the invention, or of a pharmaceutical composition comprising these microparticles, to a subject in need of said treatment.

The exosomes loaded with pre-miRNA comprising an exogenous nucleotide sequence and methods of the invention may be used in the treatment of a proliferative disease. The term ‘proliferative disease’ as used herein refers to both cancer and non-cancer disease. As such, the methods may ultimately result in the killing of cells which proliferate abnormally, such as cancerous cells, including tumour cells, and other (non-malignant) tumour cells. The pre-miRNA will deliver therapeutic exogenous nucleotide sequences for the treatment of cancer into exosomes. The invention promotes the packaging of therapeutic nucleotide sequences into exosomes, which are then delivered to the cell of a patient.

RNAs such as miRNAs and shRNAs/siRNAs can be used to target cancer genes, e.g. EGFR mutated variants in cancer. Alternatively, a guide strand for CRISPR or an anti-miRNA can also be loaded into exosomes using the pre-miRNA of the invention.

Accordingly, the invention also includes a method of treating or preventing cancer in a patient using a composition of the invention.

The term “patient” includes human and other mammalian subjects that receive either prophylactic or therapeutic treatment.

The exosomes loaded with pre-miRNA comprising an exogenous nucleotide sequence of the invention may optionally be combined with another therapeutic agent to provide a combination therapy.

### Pharmaceutical Compositions

The exosomes loaded with pre-miR-21 comprising an exogenous nucleotide sequence are useful in therapy and can therefore be formulated as a pharmaceutical composition. A pharmaceutically acceptable composition typically includes at least one pharmaceutically acceptable carrier, diluent, vehicle and/or excipient in addition to the exosomes of the invention. An example of a suitable carrier is Ringer’s Lactate solution. A thorough discussion of such components is provided in Gennaro (2000) Remington: The Science and Practice of Pharmacy. 20th edition, ISBN: 0683306472.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain minor amounts of pH buffering agents. The carrier may comprise storage media such as HYPOTHERMOSOL®, commercially available from BioLife Solutions Inc., USA. Examples of suitable pharmaceutical carriers are described in “Remington’s Pharmaceutical Sciences” by E W Martin. Such compositions will contain a prophylactically or therapeutically effective amount of a prophylactic or therapeutic exosome preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, liquid solutions or suspensions, injectable and infusible solutions. The pharmaceutical composition is preferably injectable.

Pharmaceutical compositions will generally be in aqueous form. Compositions may include a preservative and/or an antioxidant.

To control tonicity, the pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition is preferably sterile. The composition is preferably gluten free. The composition is preferably non-pyrogenic.

In a typical embodiment, the exogenous nucleotide sequence loaded exosomes of the invention are suspended in a composition comprising 6-hydroxy-2,5,7,8-tetramethylchroman-2-carboxylic acid (TROLOX®), Na⁺, K⁺, Ca²⁺, Mg²⁺, Cl⁻, H₂PO₄⁻, HEPES, lactobionate, sucrose, mannitol, glucose, dextron-40, adenosine and glutathione. Typically, the composition will not include a dipolar aprotic solvent, e.g. DMSO. Suitable compositions are available commercially, e.g. HYPOTHERMASOL®-FRS. Such compositions are advantageous as they allow the exosomes to be stored at 4° C. to 25° C. for extended periods (hours to days) or preserved at cryothermic temperatures, i.e. temperatures below -20° C. The exosomes may then be administered in this composition after thawing.

The pharmaceutical composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

The exosomes of the invention will be administered at a therapeutically or prophylactically-effective dose, which will be apparent to the skilled person. Due to the low or non-existent immunogenicity of the exosomes, it is possible to administer repeat doses without inducing a deleterious immune response.

The invention is further described with reference to the following non-limiting examples.

### EXAMPLES

### Summary of the Examples

The invention has resulted from a combination of analysis of both linear sequence and tertiary (3D) structures of pre-microRNA and pri-micro-RNA, the relative abundance of specific variants in exosomes arising from CTX Neural stem cells. Additionally the invention has resulted from distinguishing the structures and sequences that lead to higher levels of expression and up-shuttling of microRNAs in harvested exosomes. This knowledge was used to design pre-miR-21 constructs in which the sequence of any exogenous nucleotide sequence of choice (e.g. miRNA, siRNA or shRNA) could be inserted and targeted to an exosome during exosome biogenesis.

The expression vector of the invention can be exchanged for transfection with synthetically produced pre-miRNA of the invention by transfection using through electroporation, lipofection, or any other equivalent technique. The expression vector can be modulated by using different transcription factors depending on c-myc expression, phorbol 12-myristate 13-acetate (PMA), or other transcription factors with binding capacity to the endogenous sequences of MIR21 or artificial sequences able to regulate the expression of the vector or modified vector.

### Example 1: The MIR21 Vector Can Be Used to Regulate the Expression of a MIR in a Cell and Transcription Factor Dependent Manner

It is understood that MIR21 is embedded in an intronic sequence of the VMP1 gene. However its expression depends on its own regulatory elements contained in the promoter region miPPR21 as described by Fujita et. al., 2008 (FIG. 1A). This promoter region is found -3,770 to -3,337 upstream to the miR-21 hairpin.

The highlighted enhancer elements in the miPPR-21 promoter depicted in FIG. 1A are:

TATA Box: GATAAATG

AP-1: GTTAATCAn

AP-1: GATGACGCACA

AP-1: GATGACACAAGCAnAAGTCA

C/EBP: nnAnTTTGCTAATGCATT

C/EBP: TAGnTTGAGAAAnnGnCC

SRF: TCCTAATAAGGACTT

STAT3: CAGTTCTTACAGGAACTnGTG

STAT3: TGGGACTTCTGAGAAGTCATT

GC Box: nnTGGGAGGnGCCT

Ets/PU.1: TTTnTGGATAAGGATGACG

Ets/PU.1: ACTAGGGATGACA

Ets/PU.1: TACAGGAACTnG

NFl: AATTGGTTCAAACCAGTT

p53: GGnCAAGTCA

We designed a vector containing the full-length of the MIR21 gene in order to test for its capacity to express any miRNA located in the miR21 exon (FIG. 1A). We have found that the expression vector is functional in cells that do not express or express very low levels of miRNA-5p as well as in our CTX0E03 cells where we found that miR21-5p is the highest miRNA been expressed (FIGS. 1B, 1C), indicating that the vector acts independently of the cell context.

Moreover, we found that the vector can be regulated by PMA, possibly due to the regulatory sequences on its promoter as described by Fujita et. al., 2008.

We also found that the over-expression of the transcription factor c-myc can effectively increase the expression of miR21 (FIG. 1D). Therefore, the expression of the mRNA for miR21 or any other miRNA located in the miR21 Exon can be regulated in the cell line CTX0E03 by a mechanism dependent on c-myc.

Looking for other regulatory sequences that could control the transcription, stability and correct processing of the mRNA and miRNA we designed several deletion constructs of the vector by including different restriction sites. We have observed the presence of regulatory elements located in the upstream and downstream region of the mir21 exon that control its expression and could affect its processing - and therefore its loading into exosomes (FIG. 2).

### Example 2: Design and Functional Studies Using Pri-MIR-Ruby2

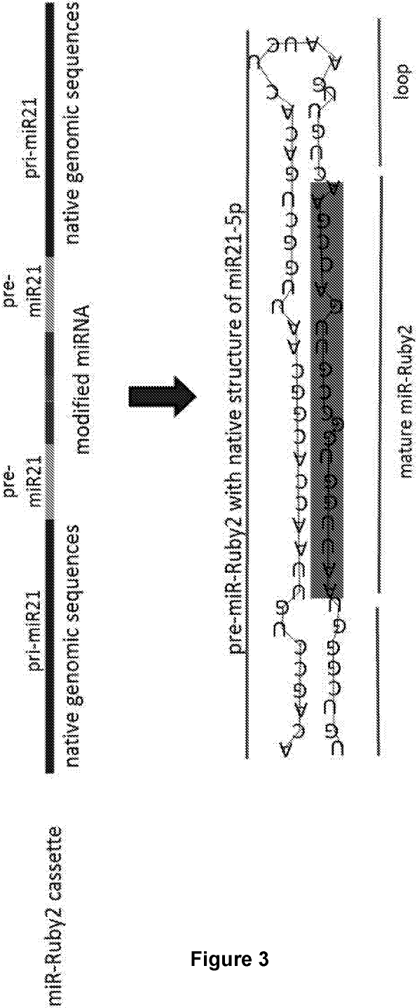

Without being bound by theory, it is understood that the Wobble pairs and structures present in the sequence of the pri-miR-21 have an important role in its folding, processing and interaction with RBPs, and therefore the sorting of the generated miRNA into exosomes (FIG. 3). To test this hypothesis, an expression vector containing the cassette pri-miR-Ruby2 was generated, which if correctly processed would generate a functional miRNA against the fluorescent protein Ruby2 (FIG. 3). This cassette was placed on the 3′ UTR region of a gene coding sequence (CDS) to facilitate its transcription by a RNA-polymerase II promoter.

In the design of the pri-miR-Ruby2, the native structure, length, G-U pairs, mismatches, deletions and loop of the pre-miR-21-5p was maintained, but not the two Wobble pairs present in the sequence of the natural mature miR21-5p, (FIG. 5B). Finally, the pre-miRNA was flanked with 5′ and 3′ terminal sequences found on the pri-miR-21-5 (FIG. 5C) and cloned the entire cassette into a lentivirus expression vector (FIG. 5D).

After electroporation of this vector into HEK293 cells, the expression of the mature miR-Ruby2 in the cytoplasm was detected by qPCR, indicating the correct generation and processing of the miRNA in the producer cell line (FIG. 6A). This miR-Ruby2 is also functional in the producer cell line, a significant down-regulation on the mRNA levels for Ruby2 in HEK293 constitutively expressing this protein was observed (FIG. 6B). Moreover, the presence of the miR-Ruby2 in purified exosomes from the culture media of producer cells was detected by qPCR in a co-culture assay (FIG. 6C). The effect of miR-Ruby2 in purified exosomes on down-regulating the expression of the protein (FIG. 6D) and mRNA (FIG. 6D) in a co-culture assay was also analysed.

### Example 3: Design of miRNAs to Be Cloned Into the Expression Vector pLVX-Exo-MIRNA

The designed vector, pLVX-Exo-miRNA, is a lentiviral vector that allows the expression of miRNAs to be loaded into exosomes based on the structure of the hsa-miR-21-5p. This vector also allows for the expression of the fluorescent protein Clover and the antibiotic selection protein for Blasticidin in order to generate stable cell lines for the expression of the desired miRNA cassette.

The miR21 cassette contains all the elements that are necessary for the expression and loading of miRNAs into exosomes.

For generating a miRNA to be cloned into the pLVX vector the skilled person would need to design a pri-miRNA based on the structure of the pri-miR21 to be cloned into the pLVX vector.

The pri-miRNA to be cloned into the pLVX vector should contain the following features:

1. 6 nucleotides corresponding to the restriction enzyme Avrll: CCTAGG;

2. 130 nucleotides corresponding to the 5′ sequence of the hsa pri-miR21:

GTTCGATCTTAACAGGCCAGAAATGCCTGGGTTTTTTTGGTTTGTTTTTG
TTTTTGTTTTTTTATCAAATCCTGCCTGACTGTCTGCTTGTTTTGCCTAC
CATCGTGACATCTCCATGGCTGTACCACCT;

3. 8 nucleotides corresponding to the 5′ sequence of the hsa-miR21-5p:

TGTCGGGT;

4. Reverse complement of the 21-nucleotide target sequence (mature miRNA sequence). This will be the stem of the miRNA once transcribed. This can be designed any of the multiple online tools available;

5. 16 nucleotides corresponding to the terminal loop of the hsa-mir-21:

CTGTTGAATCTCATGG;

6. Nucleotides 2-6 of the sense target sequence;

7. 1 nucleotide that does not match to the antisense target sequence according to Watson-Crick base pair complementation and that makes a Wobble base pair;

8. Nucleotides 8-12 and 14-21 of the sense target sequence;

9. 8 nucleotides corresponding to the 3′ sequence of the hsa-miR21-5p:

GTCTGACA;

10. 112 nucleotides corresponding to the 3′ sequence of the hsa-pri-miR-21:

TTTTGGTATCTTTCATCTGACCATCCATATCCAATGTTCTCATTTAAACA
TTACCCAGCATCATTGTTTATAATCAGAAACTCTGGTCCTTCTGTCTGGT
ACTAGTGCTAGC

; and

11. 8 nucleotides corresponding to the restriction enzyme Notl: GCGGCCGC.

Once designed, the sequence can be ordered as a double strand DNA sequence in order to be cloned directly into the expression vector of the invention using the restriction sites Avrll and Notl.

### Example 4

Further tests on the processing and loading of miRNAs into exosomes modify the vector containing the designed pri-miRNA cassette as follows:
5′ and 3′ terminal sequences of miR-21-5p: substitute these sequences for other ones from different miRNAs;
pre-miR-21-5p structure: substitute the wobble base pairs in the pre-miRNA sequence and use canonical Watson-Crick pairs;
Internal loop: substitute it with other loop sequences; and
Mismatches and substitution: remove those ones and generate fully complementary strands of the designed miRNA maintaining the original pre-miRNA sequence and loop.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### SELECTED REFERENCES

1. Bolukbasi, M.F., Mizrak, A., Ozdener, G.B., Madlener, S., Ströbel, T., Erkan, E.P., Fan, J.B., O Breakefield, X., and Saydam, O. (2012). “miR-1289 and “Zipcode″-like Sequence Enrich mRNAs in Microvesicles”. Molecular Therapy - Nucleic Acids, 1, e10.

2. Colombo, M., Raposo, G., and Théry, C. (2014). “Biogenesis, Secretion, and Intracellular Interactions of Exosomes and Other Extracellular Vesicles”. Annual Review of Cell and Developmental Biology, 30, 255-289.

3. Cullen, B.R. (2004). “Transcription and Processing of Human miRNA precursors”. Molecular Cell, 6, 861-865.

4. Fleige, S., and Pfaffl M.W. (2006). “RNA integrity and the effect on the real-time qRT-PCR performance”. Molecular Aspects of Medicine, 27, 126-139.

6. 5. Fujita, S., Ito, T., Mizutani, T., Minoguchi, S., Yamamichi, N., Sakurai, K., Iba, H., (2008). “miR-21 Gene Expression Triggered by AP-1 Is Sustained through a Double-Negative Feedback Mechanism”. The Journal of Molecular Biology, 378, 492-504.Gerbert, L.F.R., and MacRae, I.J. (2019). “Regulation of microRNA function in animals”. Nature Reviews, 20, 21-37.

7. Hodges, H., Pollock, K., Stroemer, P., Patel, S., Stevanato, L., Reuter, I., Sinden, J. (2007). “Making stem cell lines suitable for transplantation”. Cell Transplantation, 16, 101-15.

8. Janas, T., Janas, M.M., Sapón, K., and Janas, T. (2015). “Mechanisms of RNA loading into exosomes”. FEBS Letters, 589, 1391-1398.

9. Kaczmarek, J.C., Kowalski, P.S., and Anderson, G.D. (2017) “Advances in the delivery of RNA therapeutics: from concept to clinical reality”. Genome Medicine, 9:60.

10. Kanasty, R., Dorkin, J.R., Vegas, A., and Anderson, D. (2013). “Delivery materials for siRNA therapeutics”. Nature Materials, 12, 967-977.

11. Katsuda, T., Kosaka, N., Takeshita, F., and Ochiya, T. (2013) “The therapeutic potential of mesenchymal stem cell-derived extracellular vesicles”. Proteomics, 13, 1637-1653.

12. Katsuda, T., Tsuchiya, R., Kosaka, N., Yoshioka, Y., Takagaki, K., Oki, K., Takeshita, F., Sakai, Y., Kuroda, M., and Ochiya,T. (2013) “Human adipose tisse-derived mesenchymal stem cells secrete functional neprilysin-bound exosomes”. Scientific Reports, 3:1197.

13. Kornblum, H. (2007). “Introduction to Neural Stem Cells”. Stroke, 38, 810-816.

14. Littlewood, T.D., Hancock, D.C., Danielian, P.S., Parker, M.G., and Evan, G.I. (1995). “A modified oestrogen receptor ligand-binding domain as an improved switch for the regulation of heterologous proteins”. Nucleic Acid Research, 23, 1686-1690.

15. McClure, V.L., Seo, G.J., and Sullivan, C.S. (2011). “Reporter-Based Assays for Analysing RNA Interference in Mammalian Cells”. Methods in Molecular Biology, 725, 173-189.

16. Miljan, E.A., and Sinden, J.D. (2009). “Stem cell treatment of ischemic brain injury”. Current Opinion in Molecular Therapeutics, 4, 394-403.

17. Mirihana Arachchilage, G., Dassanayake, A.C., Basu, S. (2015). “A potassium ion-dependent RNA structural switch regulates human pre-miRNA 92b maturation.” Cell: Chemistry and Biology, 22, 262-272.

18. Sridharan, K., and Gogtay, N.J. (2016). “Therapeutic nucleic acids: current clinical status”. British Journal of Clinical Pharmacology, 82, 659-672.

19. Sutaria et al. J Extracell Vesicles. 2017; 6(1): 1333882 “Low active loading of cargo into engineered extracellular vesicles results in inefficient miRNA mimic delivery”

20. Tatiparti, K., Sau, S., Kashaw, S.K., and lyer, AK. (2017). “siRNA Delivery Strategies: A Comprehensive Review of Recent Developments”. Nanomaterials, 7, e77.

21. Tsutsumi, A.,Kawamata, T., Izumi, N., Seitz, H., & Tomari, Y. (2011). “Recognition of the pre-miRNA structure by Drosophila Dicer-1”. Nature Structural & Molecular Biology, 18, 1153-1158.

22. Villarroya-Beltri, C., Gutiérrez-Vázquez, C., Sánchez-Cabo, F., Pérez-Hernández, D., Vázquez, J., Martin-Cofreces, N., Martinez-Herrera, D.J., Pascual-Montano, A., Mittelbrunn, M., and Sánchez-Madrid, F. (2013). “Sumoylated hnRNPA2B1 controls the sorting of miRNAs into exosomes through binding to specific motifs”. Nature Communications, 4, e:2980.

23. Whitehead, K.A., Langer, R., and Anderson, D.G. (2009). “Knocking down barriers: advances in siRNA delivery”. Nature Reviews Drug Discovery, 8, 129-138.

24. Wilson, L.O., O’Brien, A.R., and Bauer, D.C. (2018). “The Current State and Future of CRISPR-Cas9 gRNA Design Tools”. Frontiers in Pharmacology, 9, e749.

25. Yang, J.S., Maurin, T., Robine, N., Rasmussen, K.D., Jeffrey, K.L., Chandwani, R., Papapetrou, E.P., Sadelain, M., O’Carroll, D., and Lai, E.C. (2010). “Conserved vertebrate mir-451 provides a platform for Dicer-independent, Ago2-mediated microRNA biogenesis.” PNAS, 107, 15163-15168.

26. Yoo, K.W., Li, N., Makani, V., Singh, R.N., Atala, A., and Lu, B. (2018). “Large-Scale Preparation of Extracellular Vesicles Enriched with Specific microRNA.” Tissue Engineering: Part C, 24, 637-644.

1. A pre-miRNA for targeting an exogenous nucleotide sequence to an exosome, wherein the pre-miRNA comprises the exogenous nucleotide sequence and a stem-loop structure, and wherein the stem of the stem-loop structure comprises at least one wobble pair.

2. A pre-miRNA according to claim 1, wherein the pre-miRNA comprises:
a a 5′ end comprising the at least one wobble pair; and/or
b the exogenous nucleotide sequence ; and/or
c a 3′ end comprising the loop of the stem-loop structure and at least one wobble pair.

3. A pre-miRNA according to claim 1, comprising the configuration: A-B-C, wherein:
a A is a pre-miRNA 5′ end, wherein the pre-miRNA 5′ end comprises at least 50%, 60%, 70%, 80%, 90% or greater sequence identity to the 5′ end of pre-miR-21;
b B is the exogenous nucleotide sequence and is not naturally found in pre-miR-21; and
c C is a pre-miRNA 3′ end, wherein the pre-miRNA 3′ end comprises at least 50%, 60%, 70%, 80%, 90% or greater sequence identity to the 3′ end of pre-miR-21.

4. A pre-miRNA according to claim 1, wherein the pre-miRNA has the formula:
$$\begin{matrix} & & & \text{LL} & \\ \left[\text{MNNNWNW}\right] & \left\{{\text{WN}}_{\text{x1}} - {\text{N}}_{\text{x2}} {\text{MN}}_{\text{x3}} -\right\} & \left(\text{NWWN} - \text{L}\right) & & \text{L} \\ \left[\text{Mnnnwnw}\right] & \left\{{\text{wn}}_{\text{x1}} {\text{Dn}}_{\text{x2}} {\text{mn}}_{\text{x3}} \text{D}\right\} & \left(\text{nwwnDl}\right) & & \\ & & & \text{LL} & \end{matrix}$$
wherein:
each N and n is independently any nucleotide selected such that each N hybridises with a corresponding n;
Nₓ₁, Nₓ₂, Nₓ₃, nₓ₁, nₓ₂, and nₓ₃ are each independently any nucleotide sequence selected such that Nₓ₁, Nₓ₂,and Nₓ₃ hybridise with nₓ₁, nₓ₂, and nₓ₃, respectively;
M and m are each independently a nucleotide selected such that M is mismatched with or does not hybridise with m;
each D is independently a nucleotide that is present on one side of the stem-loop structure and is not hybridised with any nucleotide of the other side of the stem-loop structure;
each W and w is independently a nucleotide selected such that each W forms a wobble pair with a corresponding w;
each L and 1 is independently a nucleotide selected such that the loop of the stem-loop structure is formed with L nucleotides, and each 1 hybridises with a corresponding L; and
wherein the pre-miRNA comprises the configuration: A-B-C, wherein:
[...] of the formula is A, a miR-21 5′ end structural mimic;
{...} of the formula is B, the exogenous nucleotide sequence; and
(...of the formula is C, a miR-21 3′ end structural mimic.

5. A pre-miRNA according to claim 4, wherein Nₓ₁ has a length from 4 to 12 nucleotides, Nₓ₂ has a length from 2 to 8 nucleotides, and Nₓ₃ has a length from 2 to 8 nucleotides.

6. A pre-miRNA according to claim 1, wherein:
the stem-loop structure has an overall length of 60 to 80 nucleotides; and/or
the pre-miRNA comprises a transcription factor-regulated promoter upstream of the exogenous nucleotide sequence.

7. A pre-miRNA according to claim 1, wherein the pre-miRNA has the formula:
$$\begin{matrix} & & & \text{LL} & \\ \left[\text{MNNNWNW}\right] & \left\{\text{WNNNNNNNN} - \text{NNNNNMNNNNN} -\right\} & \left(\text{NWWN} - \text{L}\right) & & \text{L} \\ \left[\text{Mnnnwnw}\right] & \left\{\text{wnnnnnnnnDnnnnnmnnnnnD}\right\} & \left(\text{nwwnDl}\right) & & \\ & & & \text{LL} & \end{matrix}$$
wherein:
each N and n is independently any nucleotide selected such that each N hybridises with a corresponding n
M and m are each independently a nucleotide selected such that M is mismatched with or does not hybridise with m;
each D is independently a nucleotide that is present on one side of the stem-loop structure and is not hybridised with any nucleotide of the other side of the stem-loop structure;
each W and w is independently a nucleotide such that each W forms a wobble pair with a corresponding w;
each L and 1 is independently a nucleotide selected such that the loop of the stem-loop structure is formed with L nucleotides, and each 1 hybridises with a corresponding L; and
wherein the pre-miRNA comprises the configuration: A-B-C, wherein:
[...] of the formula is A, a pre-miRNA 5′ end;
{...} of the formula is B, the exogenous nucleotide sequence; and
(...of the formula is C, a pre-miRNA 3′ end.

8. A pre-miRNA according to claim 1, comprising the sequence of pre-miR-21 and the exogenous nucleotide sequence in place of the mature miR-21 sequence.

9. A pre-miRNA according to claim 1, wherein the at least one wobble pair (W-w) comprises guanine-uracil (G-U or U-G), hypoxanthine-uracil (I-U or U-I), hypoxanthine-adenine (I-A or A-I), and/or hypoxanthine-cytosine (I-C or C-I).

10. A pre-miRNA according to claim 1, wherein the pre-miRNA has the formula:
$$\begin{matrix} & & & \text{UC} & \\ \left[\text{ACAGUCU}\right] & \left\{\text{GNNNNNNNN} - \text{NNNNNMNNNNN} -\right\} & \left(\text{GGUA} - \text{C}\right) & & \text{U} \\ \left[\text{UGUCGGG}\right] & \left\{\text{UnnnnnnnnDnnnnnmnnnnnD}\right\} & \left(\text{CUGUUG}\right) & & \\ & & & \text{AA} & \end{matrix}$$
wherein:
each N and n is independently any nucleotide selected such that each N hybridises with a corresponding n;
M and m are each independently a nucleotide selected such that M is mismatched with or does not hybridise with m;
D is a nucleotide that is present on one side of the stem-loop structure and not hybridised with any nucleotide of the other side of the stem-loop structure; and wherein the pre-miRNA comprises the configuration: A-B-C, wherein:
[...] of the formula is A, a pre-miRNA 5′ end;
{...} of the formula is B, the exogenous nucleotide sequence; and
(...of the formula is C, a pre-miRNA 3′ end.

11. A pre-miRNA according to claim 1, wherein the exogenous nucleotide sequence is an siRNA, miRNA, anti-miR, antisense oligonucleotide (ASO), or CRISPR guide strand sequence.

12. A pre-miRNA according to claim 1, wherein the exogenous nucleotide sequence is targeted to the exosome during exosome biogenesis.

13. A pre-miRNA according to claim 1, wherein the exogenous nucleotide sequence modulates gene activity in a target cell.

14. A cassette comprising a pre-miRNA according to claim 1, said cassette comprising, in the following order:
a a 5′ pri-miR-21 sequence;
b the pre-miRNA according to claim 1; and
c a 3′ pri-miR-21 sequence.

15. A vector comprising a cassette according to claim 14.

16. A cell comprising a vector according to claim 15.

17. A cell according to claim 16, wherein the cell is a stem .

18. A cell according to claim 16, wherein the pre-miRNA is present within exosomes in the cell.

19. A method of loading exosomes with an exogenous nucleotide sequence, the method comprising producing exosomes from a cell according to claim 16.

20. A method of preparing exosomes, the method comprising:
a culturing cells according to claim 16; and
b harvesting conditioned media from the culturing of the cells.

21. An exosome obtainable or obtained by the method of claim 19.

22. A method of delivering an exogenous nucleotide sequence to a target cell, the method comprising:
contacting the target cell with an exosome according to claim 21.

23. A method of modulating gene activity in a target cell, the method comprising administering to the target cell an exosome according to claim 21.

24. A pharmaceutical composition comprising an exosome according to claim 21.

25. An exosome according to claim 21, for use in therapy.

26. Use of one or more of the regulatory elements located in the upstream or downstream region of the mir21 exon to control miRNA loading into an exosome according to claim 21.


SEQUENCE LISTING (24 sequences)

SEQ ID NO: 1 - RNA, 16 nt, Homo sapiens
gguacucuaaguuguc

SEQ ID NO: 2 - RNA, 72 nt, Homo sapiens
ugucggguagcuuaucagacugauguugacuguugaaucucauggcaacaccagucgaugggcugucugaca

SEQ ID NO: 3 - RNA, 22 nt, Homo sapiens
uagcuuaucagacugauguuga

SEQ ID NO: 4 - RNA, 21 nt, Homo sapiens
caacaccagucgaugggcugu

SEQ ID NO: 5 - DNA, 11 bp, Homo sapiens
gatgacgcaca

SEQ ID NO: 6 - DNA, 20 bp, Homo sapiens
  misc_feature (14)..(14): n is a, c, g, or t
gatgacacaagcanaagtca

SEQ ID NO: 7 - DNA, 18 bp, Homo sapiens
  misc_feature (1)..(2): n is a, c, g, or t
  misc_feature (4)..(4): n is a, c, g, or t
nnantttgctaatgcatt

SEQ ID NO: 8 - DNA, 18 bp, Homo sapiens
  misc_feature (4)..(4): n is a, c, g, or t
  misc_feature (13)..(14): n is a, c, g, or t
  misc_feature (16)..(16): n is a, c, g, or t
tagnttgagaaanngncc

SEQ ID NO: 9 - DNA, 15 bp, Homo sapiens
tcctaataaggactt

SEQ ID NO: 10 - DNA, 21 bp, Homo sapiens
  misc_feature (18)..(18): n is a, c, g, or t
cagttcttacaggaactngtg

SEQ ID NO: 11 - DNA, 21 bp, Homo sapiens
tgggacttctgagaagtcatt

SEQ ID NO: 12 - DNA, 14 bp, Homo sapiens
  misc_feature (1)..(2): n is a, c, g, or t
  misc_feature (10)..(10): n is a, c, g, or t
nntgggaggngcct

SEQ ID NO: 13 - DNA, 19 bp, Homo sapiens
  misc_feature (4)..(4): n is a, c, g, or t
tttntggataaggatgacg

SEQ ID NO: 14 - DNA, 13 bp, Homo sapiens
actagggatgaca

SEQ ID NO: 15 - DNA, 12 bp, Homo sapiens
  misc_feature (11)..(11): n is a, c, g, or t
tacaggaactng

SEQ ID NO: 16 - DNA, 18 bp, Homo sapiens
aattggttcaaaccagtt

SEQ ID NO: 17 - DNA, 10 bp, Homo sapiens
  misc_feature (3)..(3): n is a, c, g, or t
ggncaagtca

SEQ ID NO: 18 - DNA, 130 bp, Homo sapiens
gttcgatcttaacaggccagaaatgcctgggtttttttggtttgtttttgtttttgtttttttatcaaatcctgcctgactgtctgcttgttttgcctaccatcgtgacatctccatggctgtaccacct

SEQ ID NO: 19 - DNA, 16 bp, Homo sapiens
ctgttgaatctcatgg

SEQ ID NO: 20 - DNA, 112 bp, Homo sapiens
ttttggtatctttcatctgaccatccatatccaatgttctcatttaaacattacccagcatcattgtttataatcagaaactctggtccttctgtctggtactagtgctagc

SEQ ID NO: 21 - DNA, 474 bp, Homo sapiens
  misc_feature (1)..(1): n is a, c, g, or t
  misc_feature (3)..(5): n is a, c, g, or t
  misc_feature (7)..(7): n is a, c, g, or t
  misc_feature (12)..(13): n is a, c, g, or t
  misc_feature (16)..(20): n is a, c, g, or t
  misc_feature (22)..(22): n is a, c, g, or t
  misc_feature (28)..(28): n is a, c, g, or t
  misc_feature (30)..(34): n is a, c, g, or t
  misc_feature (36)..(36): n is a, c, g, or t
  misc_feature (39)..(40): n is a, c, g, or t
  misc_feature (42)..(42): n is a, c, g, or t
  misc_feature (47)..(49): n is a, c, g, or t
  misc_feature (53)..(54): n is a, c, g, or t
  misc_feature (58)..(64): n is a, c, g, or t
  misc_feature (66)..(67): n is a, c, g, or t
  misc_feature (69)..(70): n is a, c, g, or t
  misc_feature (78)..(78): n is a, c, g, or t
  misc_feature (81)..(81): n is a, c, g, or t
  misc_feature (83)..(83): n is a, c, g, or t
  misc_feature (85)..(85): n is a, c, g, or t
  misc_feature (89)..(89): n is a, c, g, or t
  misc_feature (93)..(93): n is a, c, g, or t
  misc_feature (96)..(96): n is a, c, g, or t
  misc_feature (102)..(102): n is a, c, g, or t
  misc_feature (110)..(113): n is a, c, g, or t
  misc_feature (116)..(117): n is a, c, g, or t
  misc_feature (125)..(125): n is a, c, g, or t
  misc_feature (133)..(133): n is a, c, g, or t
  misc_feature (138)..(142): n is a, c, g, or t
  misc_feature (145)..(146): n is a, c, g, or t
  misc_feature (158)..(159): n is a, c, g, or t
  misc_feature (181)..(187): n is a, c, g, or t
  misc_feature (193)..(195): n is a, c, g, or t
  misc_feature (203)..(203): n is a, c, g, or t
  misc_feature (208)..(208): n is a, c, g, or t
  misc_feature (210)..(211): n is a, c, g, or t
  misc_feature (213)..(213): n is a, c, g, or t
  misc_feature (232)..(232): n is a, c, g, or t
  misc_feature (274)..(274): n is a, c, g, or t
  misc_feature (283)..(284): n is a, c, g, or t
  misc_feature (286)..(286): n is a, c, g, or t
  misc_feature (290)..(290): n is a, c, g, or t
  misc_feature (292)..(292): n is a, c, g, or t
  misc_feature (296)..(296): n is a, c, g, or t
  misc_feature (301)..(301): n is a, c, g, or t
  misc_feature (305)..(306): n is a, c, g, or t
  misc_feature (310)..(310): n is a, c, g, or t
  misc_feature (318)..(322): n is a, c, g, or t
  misc_feature (329)..(329): n is a, c, g, or t
  misc_feature (364)..(364): n is a, c, g, or t
  misc_feature (411)..(411): n is a, c, g, or t
  misc_feature (451)..(451): n is a, c, g, or t
  misc_feature (460)..(460): n is a, c, g, or t
  misc_feature (464)..(465): n is a, c, g, or t
  misc_feature (467)..(467): n is a, c, g, or t
  misc_feature (473)..(473): n is a, c, g, or t
ngnnntntgggnnagnnnnngnagctcngnnnnnanaanntnatcannnatcnntatnnnnnnnanntnngaacaagntgntnantaangtantgnagtttntgtgcaannnntcnnccataaancatgaaangattnnnnncanngttcatagttcnntctttgttcctttgttaatcannnnnnnctgacnnntgggaggngcctncnnantttgctaatgcattctttntggataaggatgacgcacagattgtcctaataaggacttagnttgagaaanngnccgncncctntgagnaganngggncaagtcannnnnagggcgngcagtttcttttttaactagggatgacacaagcanaagtcatttccttattaattggttcaaaccagttcttacaggaactngtggtgataaatgtgggacttctgagaagtcattcatttnattctttgngccnnancagagna

SEQ ID NO: 22 - RNA, 72 nt, Homo sapiens
acagccuguuaaccacggcaauuggcugacacucuaaguugucaagccaguugccggugguuaaugggcugu

SEQ ID NO: 23 - RNA, 72 nt, Homo sapiens
acagucugucggguagcugaccacaacgguacucuaaguugucaguuguagucagacuauucgaugggcugu

SEQ ID NO: 24 - DNA, 72 bp, Homo sapiens
acagucugaactcaaggtacacgccuagguacucuaaguugucuuaggcauguacccuugaguuugggcugu